(19) United States
(12) Patent Application Publication
(10) Pub. No.: US 2005/0238666 A1
(43) Pub. Date: Oct. 27, 2005

(54) Methods of enhancing stem cell engraftment

(76) Inventors: David A. Williams, Cincinnati, OH (US); Gabriel Ghiaur, Blue Ash, OH (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/003,935

(22) Filed: Dec. 3, 2004

Related U.S. Application Data
(60) Provisional application No. 60/527,589, filed on Dec. 5, 2003.

(51) Int. Cl. A61K 31/56; A61K 39/08; A61K 31/401; A61K 31/366; A61K 31/225

(52) U.S. Cl. 424/239.1; 514/460; 514/548; 514/423; 514/171

(57) ABSTRACT

The present invention provides improved methods and pharmaceutical compositions for enhancing stem cell engraftment, comprising the administration of an effective amount of a modulator of RhoGTPases.

### CROSS REFERENCE TO RELATED APPLICATIONS

This patent application claims priority under 35 U.S.C. § 119(e) to U.S. Provisional Patent No. 60/527,589, entitled “Methods of Enhancing Stem Cell Engraftment,” filed Dec. 5, 2003, which is incorporated herein by reference in its entirety.

### Statement Regarding Federally Sponsored Research

This invention was made in part with Government support under Grant No. R01DK62757, awarded by the National Institutes of Health. The Government may have certain rights in this invention.

### FIELD OF THE INVENTION

The present invention provides improved methods and pharmaceutical compositions for enhancing stem cell engraftment, comprising the administration of an effective amount of a modulator of RhoGTPases.

### BACKGROUND OF THE INVENTION

The various mature blood cell types are all ultimately derived from a single class of progenitor cell known as hematopoietic stem cells. True stem cells are both pluripotent—that is they can give rise to all cell types—and capable of self-renewal. This is defined by their ability to repopulate an individual whose hematopoietic system has been destroyed by radiation. Stem cells represent a very small percentage of bone marrow cells, and are normally quiescent. When stimulated to divide, they give rise to additional stem cells or more committed, differentiated daughter cells with less proliferative potential. The term stem cell is often also applied to these so-called “early progenitor” cells. Sequential rounds of division and differentiation give rise to an enormous amplification of cell numbers, necessary for the production of mature blood cells. This process of division and differentiation is subject to regulation at many levels to control cell production.

Leukocytes are derived from hematopoietic stem cells and are important in maintaining the body's defense against disease. For example, macrophages and lymphocytes are involved in potentiating the body's response to infection and tumors; granulocytes (neutrophils, eosinophils and basophils) are involved in overcoming infection, parasites and tumors. Other cell types derived from hematopoietic stem cells include platelets and erythrocytes.

All of mammalian differentiated blood cells are derived from stem cells. In vivo, the stem cell is able to self-renew, so as to maintain a continuous source of pluripotent cells. In addition, when subject to particular environments and/or factors, the stem cells can differentiate to yield dedicated progenitor cells, that in turn can serve as the ancestor cells to a limited number of blood cell types. These ancestor cells will go through a number of stages before ultimately yielding mature cells.

### SUMMARY OF THE INVENTION

One embodiment provides for Rho modulators. The disclosed method relates to the novel use of an active compound to produce a pharmaceutical preparation for enhanced engraftment of hematopoietic stem cells in a subject. In some embodiments a pharmaceutical packaging unit containing an active compound and informational instructions regarding the application of the modulator is provided. The modulator can be one or more active compounds or a combination of other agents for enhanced engraftment of stem cells.

Further embodiments include the use of Rho, or proteins related to Rho as therapeutic targets for agents designed to enhance stem cell engraftment. One embodiment pertains to the use of Rho antagonists or modulators that foster stem cell engraftment. The therapeutic agent, modulator or antagonist can include, but are not limited to, small molecules, proteins, antibodies, nucleic acids or peptides, or any agent that binds to Rho or its family members to modulate this pathway, including genes delivered by way of vector and/or plasmid irradiated gene transfer.

Another embodiment pertains to the use of the Rho regulatory pathway as a target for Rho antagonists. This pathway involves the GDP/GTP exchange factors (GEFs). Rho has two interconvertible forms, GDP-bound inactive, and GTP-bound active forms. The GEPs promote the exchange of nucleotides and thereby constitute targets for regulating the activity of Rho. In another embodiment GDP dissociation inhibitors (GDIs) inhibit the dissociation of GDP from Rho, and thereby prevent the binding of GTP necessary for the activation of Rho. Therefore, GDIs are targets for agents that regulate Rho activity. The GTP-bound active Rho can be converted to the GDP-bound inactive form by a GTPase reaction that is facilitated by its specific GTPase activating protein (GAP). Thus, another embodiment pertains to the use of GAPs as targets for the regulation of Rho activity. Another embodiment pertains to the fact that Rho is found in the cytoplasm complexed with a GTPase inhibiting protein (GDI). To become active, Rho binds GTP and is translocated to the membrane. Thus, agents that affect Rho binding to the plasma membrane are also considered within the scope of this invention. Yet another embodiment pertains to the observation that a bacterial mon-ADP ribosyltransferase, C3 transferase, ribosylates Rho to inactivate the protein. Thus this embodiment pertains to the use of C3 transferase to inactivate Rho and stimulate stem cell engraftment. Likewise, other bacterial toxins, such as toxins A and B, with related Rho-inhibitory activity are considered to be within the scope of the method herein. Moreover, various mutations of the Rho protein can create dominant negative Rho, that can interfere with the biological activity of endogenous Rho in stem cells. Thus, yet a further embodiment pertains to the use of dominant negative forms of Rho, used to inactivate Rho, to foster stem cell engraftment.

A method is provided herein for enhancing the regeneration of hematopoietic tissue through improved stem cell transplantation using a Rho modulator. The method for enhancing stem cell engraftment comprises administering to an individual in need thereof (i) a stem cell graft and (ii) a Rho modulator, wherein the Rho modulator is administered in an amount effective to promote engraftment of the bone marrow in the individual.

The method is useful to enhance the effectiveness of stem cell transplantation as a treatment for cancer. The treatment of cancer by x-irradiation or alkylating therapy destroys the bone marrow microenvironment as well as the hematopoietic stem and progenitor cells. The current treatment is to transplant the patient after marrow ablation with stem cells that has been previously harvested from bone marrow, as peripheral blood or umbilical cord blood of the donor.

Modes of administration of the Rho modulator include but are not limited to systemic intravenous injection and injection directly to the intended site of activity. The active agent can be administered by any convenient route, for example by infusion or bolus injection and can be administered together with other biologically active agents. Administration is preferably systemic.

The disclosure also provides pharmaceutical compositions. Such compositions comprise a therapeutically effective amount of the Rho modulator, and a pharmaceutically acceptable carrier or excipient. Such a carrier includes but is not limited to cell medium plus serum albumin, saline, buffered saline, dextrose, water, and combinations thereof. The formulation should suit the mode of administration.

The method can be altered, particularly by (1) increasing or decreasing the time interval between injecting the Rho modulator and implanting the tissue; (2) increasing or decreasing the amount of active agent and/or cells injected; (3) varying the number of injections; (4) varying the method of delivery of active agent and cells; or (5) varying the source of cells. Although cells derived from the subject or allogeneic donors are preferable, the cells can be obtained from other individuals or species, or from genetically engineered inbred donor strains, or from in vitro cell culture.

Methods are provided for autologous hematopoietic stem cell transplantation in patients undergoing cytoreductive therapies, and particularly to methods in which bone marrow, peripheral blood stem cells or umbilical cord blood cells are removed from a patient prior to myelosuppressive cytoreductive therapy or removed from a normal donor, expanded in ex vivo culture in the presence of a Rho modulator, and optionally a growth factor, and then re-administered to the patient concurrent with or following cytoreductive therapy to counteract the myelosuppressive effects of such therapy. The transplantation can also be for treatment of genetic disorders, heart disease and CNS disorders.

Accordingly, autologous stem cell transplantation has proven to be a valuable technique to speed recovery from cytoreductive therapies. Improvements in autologous hematopoietic cell transplantation can further speed recovery from cytoreductive therapies and even allow the use of higher and more effective doses in cytoreductive therapies. The methods are an improvement in autologous hematopoietic cell transplantation.

In one aspect, methods and kits are provided for enhanced engraftment of hematopoietic progenitor cells comprising the administration of the active compounds herein to a patient in need of such treatment.

According to another embodiment, the active compound(s) can be administered after the administration of therapeutic or chemotherapeutic agents in order to enhance the engraftment of hematopoietic stem cells.

Further aspects and advantages will be disclosed in the following examples, which should be regarded as illustrative and not limiting the scope of this application.

### BRIEF DESCRIPTION OF THE DRAWINGS

This invention, as defined in the claims, can be better understood with reference to the following drawings. The drawings are not necessarily to scale, emphasis instead being placed upon clearly illustrating principles of the present methods and compounds.

FIG. 1 is a graphical depiction of the constructs used to test the function of Rho and Cdc42 in the engraftment of stem cells where (a) shows the control vector and (b) shows the vector where Rac2D57N, RhoAN19 and Cdc42N17 cDNAs are cloned into a modified murine stem cell virus-based bicistronic vector.

FIG. 2 is a graphical representation of the experimental design showing that mice are injected with bone marrow cells.

FIG. 3 is an immunoblot showing the expression of the transgene in the transplanted cells where (a) shows the Flag-Rac2D57N and control vectors and (b) shows the HA-RhoAN19, HA-Cdc42N17 and control vectors.

FIG. 4 is a graphical representation of the pull-down assay for RhoGTPases as described herein.

FIG. 5 shows Immunoblot results of the function of the transgenes in peripheral blood cells stimulated with agonists where (a) shows the effects of PDGF stimulation on RacGTPase for control and Rac2D57N cells; (b) shows the effects of LPA stimulation on RhoGTPase for control and RhoAN19 cells; and (c) shows the effects of bradikinin stimulation on Cdc42GTP for control and Cdc42N17 cells.

FIG. 6 is a graphical representation of the EGFP+ peripheral blood cells at 3, 7, 11, and 16 weeks for MIEG3, RhoAN19, Rac2D57N, and Cdc42N17 transplanted animals.

FIG. 7 is an immunoblot showing the expression of the transgene in the bone marrow of the mice at 16 weeks post-transplantation where (a) shows the Flag-Rac2D57N and control vectors and (b) shows the HA-RhoAN19, HA-Cdc42N17 and control vectors.

FIG. 8 is a graphical representation of EGFP+ cells from bone marrow, spleen and peripheral blood at 16 weeks for MIEG3, RhoAN19, Rac2D57N, and Cdc42N17 and control vectors.

FIG. 9 is a graphical representation of EGFP+ progenitor cells (CFU's) pre-transplant and at 16 weeks for MIEG3, RhoAN19, Rac2D57N, and Cdc42N17 and control vectors

FIG. 10 is a graphical representation of the EGFP+ peripheral blood cells at 3, 7, 11, and 16 weeks for MIEG3, RhoAN19, Rac2D57N, and Cdc42N17 transplanted.

### DETAILED DESCRIPTION OF THE PREFERRED EMBODIMENT

Unless defined otherwise, all technical and scientific terms used herein have the same meanings as commonly understood by one of ordinary skill in the art. Although any methods and materials similar or equivalent to those described herein can be used in the practice or testing of the methods and compounds, the preferred methods, devices, and materials are now described. All references, publications, patents, patent applications, and commercial materials mentioned herein are incorporated herein by reference for the purpose of describing and disclosing the cell lines, vectors, and methodologies which are reported in the publications which might be used in connection with the methods and compounds.

Throughout this document, all temperatures are given in degrees Celsius, and all percentages are weight percentages unless otherwise stated. The following are definitions of terms used in this specification. The initial definition provided for a group or term herein applies to that group or term throughout the present specification, individually or as part of another group, unless otherwise indicated. The following definitions, unless otherwise defined, apply:

The term “active agents” or “active compounds” refers to any compound or combination of compounds capable of selectively inhibiting RhoGTPase. In some embodiments, the compound is one that specifically modulates and/or inhibits one or more of RhoA, RhoB and RhoC in stem cells or progenitor cells to effect stem cell engraftment. In a further embodiment, the active agents or a combination of agents, including, e.g., growth factors and other engraftment enhancing agents, are pharmaceutically-acceptable agents for enhanced engraftment of hematopoietic stem cells in the treatment of diseases requiring peripheral stem cell transplantation. Alternatively, the active compound could comprise two or more compounds that specifically modulate and/or inhibit one or more of RhoA, RhoB and RhoC.

“Antagonist” refers to a pharmaceutical agent that inhibits and/or modulates at least one biological activity normally associated with at least one of the Rho family's 25 members, resulting in enhanced stem cell engraftment. Antagonists that can be used herein include without limitation, one or more Rho family member fragments, derivatives of Rho family members or of Rho family member fragments, analogs of Rho family members or of Rho family member fragments or of said derivative, and pharmaceutical agents, and said antagonists are further characterized by the property of modulating and/or suppressing Rho family member-mediated stem cell engraftment up to 100%. Antagonists include, but are not limited to, mutated forms of Rho, such as Rho having the effector domain has been mutated to prevent GTP exchange; the ADP-ribosyl transferase C3 and biologically effective fragments that antagonize Rho family members in one of the assays disclosed herein; and compounds such as Y-27632 that antagonise Rho-associated kinase (Somiyo, 1997, Nature, 389:908-910; Uehata, et al., 1997, Nature 389:990-994; U.S. Pat. No. 4,997,834, all of which are herein incorporated by reference in their entirety). The antagonist of Rho family members in accordance with the methods herein is not limited to Rho family members or its derivatives, but also includes the therapeutic application of all agents, referred herein as pharmaceutical agents, that alter the biological activity of the Rho family members protein such that stem cell engraftment is enhanced.

“Carriers” as used herein include pharmaceutically acceptable carriers, excipients, or stabilizers that are generally nontoxic to the cell or mammal being exposed thereto at the dosages and concentrations employed. Often the physiologically acceptable carrier is an aqueous pH buffered solution. Examples of physiologically acceptable carriers include, but are not limited to, buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG), and PLURONICS.

“Dominant negative” is commonly used to describe a gene or protein that has a dominant effect similar to that described genetically, e.g., one copy of the gene gives a mutant phenotypic effect, and a negative effect in that it prevents or has a negative impact on a biological process such as a signal transduction pathway. Dominant negative mutations, by which the mutation causes a protein to interfere with the normal function of a wild-type copy of the protein, and that can result in loss-of-function or reduced-function phenotypes in the presence of a normal copy of the gene, can be made using known methods.

By “dominant negative Rho” or “dnRho” is meant any polypeptide with at least 50%, 70%, 80%, 90%, 95%, or even 99% sequence identity to human Rho, that maintains binding affinity toward wild-type Rho but dimerization results in a kinase-inactive product. A skilled artisan will recognize that any insertion, deletion, or other mutation that imparts these properties, will function in an equivalent manner in the methods and compositions herein. An example of a dominant negative Rho protein is the mouse protein RhoAN19. This protein has a threonine (T) to Asparagine (N) point mutation at position number 19 of the 193 amino acid native RhoA protein (GenBank Accession No. AAC23710). Similarly, dominant negative proteins can be constructed for the human RhoA protein (GenBank Accession No. AAA27776) using well-known methods in the art.

An “effective amount” or “therapeutically effective amount” of an active agent disclosed herein is in preferred embodiments an amount capable of modulating, to some extent, the engraftment of stem cells. As used herein, the term “therapeutically effective amount” means generally the total amount of each active component of the pharmaceutical composition or method that is sufficient to show a meaningful patient benefit, e.g., increasing engraftment of stem cells or a reduction in aberrant conditions. When applied to an individual active ingredient, administered alone, the term refers to that ingredient alone. When applied to a combination, the term refers to combined amounts of the active ingredients that result in the therapeutic effect, whether administered in combination, serially or simultaneously. An “effective amount” can be determined empirically and in a routine manner.

A protein or polypeptide sequence of a Ras-related protein includes variants or fragments thereof obtained from any species, particularly mammalian, including bovine, ovine, porcine, murine, equine, and preferably human, from any source whether natural, synthetic, semi-synthetic, or recombinant.

The Rho family of genes is a sub-family of low molecular weight GTPases and is related to each other based on sequence homology and function (Vojtek, A. B., and Cooper, J. A., Cell 1995, 82, 527-529). Other sub-families include Ras, Rab, Arf, and Ran. As GTPases, these proteins bind and hydrolyze GTP. In an active state, they bind to GTP and transduce signals of other proteins in signal transduction pathways. In their inactive state, they are bound to GDP.

A “Rho GTPase” is generally a small, Ras-related GTP-binding protein that functions by binding and hydrolyzing GTP. Rho GTPases function as molecular switches, cycling between an inactive GDP-bound conformation and an active GTP-bound conformation and include RhoA, RhoB, RhoC, Cdc42, Rac1, Rac2, Rac3, TC10, RhoG, RhoD, Chp, WRCH1, TCL, and RIF. The terms “RhoGTPase” or “Rho GTPase protein polypeptide” refer to a protein or polypeptide sequence of a Ras-related GTP-binding protein, variants or fragments thereof obtained from any species, particularly mammalian, including bovine, ovine, porcine, murine, equine, and preferably human, from any source whether natural, synthetic, semi-synthetic, or recombinant.

By “Rho fusion gene” is preferably meant a Rho promoter and/or all or part of a Rho or dnRho coding region operably linked to a second, heterologous nucleic acid sequence. In preferred embodiments, the second, heterologous nucleic acid sequence is a reporter gene, that is, a gene whose expression can be assayed; reporter genes include, without limitation, those encoding glucuronidase (GUS), luciferase, chloramphenicol transacetylase (CAT), green fluorescent protein (GFP), alkaline phosphatase, and β-galactosidase.

Administration “in combination with” one or more additional therapeutic agents includes simultaneous (concurrent) and consecutive administration in any order.

A “liposome” is generally a small vesicle composed of various types of lipids, phospholipids and/or surfactant that is useful for delivery of a drug (such as a Rho modulator) to a mammal. The components of the liposome are commonly arranged in a bilayer

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

As used herein the term “modulator” generally means a substance or compound that modifies or changes the activity, expression or function of that which it modulates. The amount of modulation can vary considerable. In some embodiments the term “modulate” refers to a detectable alteration in an observable enzymatic activity of the target enzyme. Generally, the alteration is an inhibition of at least about 5%. The alteration is preferably an inhibition of at least 10%, 15%, 20%, 25%, 30%, 35%, 40%, 45%, 50%, 60%, 80%, 90%, 95%, or 100% as measured by any standard technique.

A “Rho inhibitor” means a compound capable of inhibiting the activity of a Rho protein, in vitro or in vivo. Such inhibition can be accomplished directly (i.e., by binding directly to Rho in a way that modulates one or more biological activities) or indirectly (for example, by modifying or interfering with a Rho ligand that in turn modulates Rho activity). Inhibition may be complete or partial and includes, but is not limited to, 100%, 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, and 0.5%.

“Treatment” is generally an intervention performed with the intention of preventing the development or altering the pathology of a disorder. Accordingly, “treatment” can refer to any or all of: therapeutic treatment and prophylactic or preventative measures or other measures. Those in need of treatment include those already with the disorder as well as those in which the disorder is to be prevented. Specifically, treatment can refer to enhancing the engraftment of stem cells by contacting stem cells with an effective amount of an appropriate Rho modulator.

### Stem Cell Engraftment

Embodiments of the invention provide for RhoGTPase-specific modulators and relates to the novel use of an active compound or a combination of therapeutic agents to produce a pharmaceutical preparation for enhanced engraftment of hematopoietic stem cells in the treatment of diseases requiring stem cell transplantation as is the case, e.g., in high-dosage chemotherapy or bone marrow ablation by irradiation. In addition, embodiments are directed to a pharmaceutical packaging unit containing an active compound and informational instructions regarding the application of the an active compound or a combination of therapeutic agents for enhanced engraftment of hematopoietic stem cells.

In one aspect, methods and kits are provided for increasing progenitor and stem cell survival and engraftment comprising the administration of RhoGTPase modulators (hereinafter referred to as “active compounds”).

In another aspect, methods and kits are provided for engraftment of hematopoietic stem and progenitor cells from peripheral blood into bone marrow comprising the administration of the active compounds to a patient in need of such treatment. The engrafted stem cells, for example, can be used to treat a patient after chemotherapy. In some embodiments, administration begins within 48 hours of stem cell transplantation. In another embodiment, the mammal is subjected to chemotherapy or radiation therapy prior or subsequent to the transplantation of the stem cells.

Preferred methods are also particularly suitable for those patients in need of repeated or high doses of chemotherapy. For some cancer patients, hematopoietic toxicity frequently limits the opportunity for chemotherapy dose escalation. Repeated or high dose cycles of chemotherapy can be responsible for severe stem cell depletion leading to important long-term hematopoietic sequelae and marrow exhaustion. The methods of preferred embodiments provide for improved mortality and blood cell count when used in conjunction with chemotherapy.

The bone marrow graft can include any cells or tissue known to one of skill in the art, including but not limited to any stem or progenitor cells and any hematopoietic stem cells as well as more committed, differentiated hematopoietic daughter cells with less proliferative potential.

Further aspects and advantages will be disclosed in the following examples, that should be regarded as illustrative and not limiting the scope of this application.

### Farnesyl Protein Transferase Inhibitors (FPTase Inhibitors)

In some embodiments, the active compound comprises one or more modulator of farnesyl protein transferase (FPTase), prenyl-protein transferase or geranylgeranyl-protein transferase as described in U.S. Pat. Nos. 6,572,850; 6,458,783; 6,423,751; 6,387,926; 6,242,433; 6,191,147; 6,166,067; 6,156,746; 6,083,979; 6,011,029; 5,929,077; 5,928,924; 5,843,941; 5,786,193; 5,629,302; 5,618,964; 5,574,025; 5,567,841; 5,523,430; 5,510,510; 5,470,832; 5,447,922, 6,596,735; 6,586,461; 6,586,447; 6,579,887; 6,576,639; 6,545,020; 6,539,309; 6,535,820; 6,528,523; 6,511,800; 6,500,841; 6,495,564; 6,492,381; 6,458,935; 6,451,812; 6,441,017; 6,440,989; 6,440,974; 6,432,959; 6,426,352; 6,410,541; 6,403,581; 6,399,615; 6,387,948; 6,387,905; 6,387,903; 6,376,496; 6,372,747; 6,362,188; 6,358,968; 6,329,376; 6,316,462; 6,294,552; 6,277,854; 6,268,394; 6,265,382; 6,262,110; 6,258,824; 6,248,756; 6,242,458; 6,239,140; 6,228,865; 6,228,856; 6,225,322; 6,218,401; 6,214,828; 6,214,827; 6,211,193; 6,194,438, each of which is specifically incorporated herein by reference in its entirety.

A “farnesyl protein transferase inhibitor” or “FPT inhibitor” or “FTI” is defined herein as a compound that: (i) potently inhibits FPT (but in many embodiments not geranylgeranyl protein transferase I) and (ii) blocks intracellular farnesylation of ras. FPT catalyzes the addition of an isoprenyl lipid moiety onto a cysteine residue present near the carboxy-terminus of the Ras protein. This is the first step in a post-translational processing pathway that is essential for both Ras membrane-association and Ras-induced oncogenic transformation. A number of FPT inhibitors have been reported, including a variety of peptidomimetic inhibitors as well as other small molecule inhibitors.

Farnesyl transferase inhibitors generally fall into two classes: analogs of farnesyl diphosphate; and protein substrates for farnesyl transferase. Farnesyl transferase inhibitors have been described in U.S. Pat. No. 5,756,528, U.S. Pat. No. 5,141,851, U.S. Pat. No. 5,817,678, U.S. Pat. No. 5,830,868, U.S. Pat. No. 5,834,434, and U.S. Pat. No. 5,773,455, incorporated herein by reference. Among the farnesyl transferase inhibitors shown to be effective for inhibiting the transfer of the farnesyl moiety to Ras-related proteins are L-739,749 (a peptidomimetic analog of the C-A-A-X sequence), L-744,832 (a peptidomimetic analog of the C-A-A-X sequence), SCH 44342 (1-(4-pyridylacetyl)-4-(8-chloro-5,6 dihydro-IIH benzo[5,6]cyclohepta[1,2-b]pyridin-11-yhdene)piperidine), BZA-5B (a benzodiazepine peptidomimetic), FTI-276 (a C-A-A-X peptidomimetic), and B1086 (a C-A-A-X peptidomimetic). Administration of farnesyl transferase inhibitors (FTIs) is accomplished by standard methods known to those of skill in the art, most preferably by administration of tablets containing the FTI, and is expected to fall approximately within a range of about 0.1 mg/kg of body to weight to about 20 mg/kg of body weight per day.

However, classes of compounds that can be used as the FPT inhibitor include, but are not limited to, fused-ringed tricyclic benzocycloheptapyridines, oligopeptides, peptido-mimetic compounds, farnesylated peptido-mimetic compounds, carbonyl piperazinyl compounds, carbonyl piperidinyl compounds, farnesyl derivatives, and natural products and derivatives.

Examples of compounds that are FPT inhibitors and the documents directed to those compounds are given below.

Fused-ring tricyclic benzocycloheptapyridines: WO 95/10514; WO 95/10515; WO 95/10516; WO 96/30363; WO 96/30018; WO 96/30017; WO 96/30362; WO 96/31111; WO 96/31478; WO 96/31477; WO 96/31505; WO 97/23478; International Patent Application No. PCT/US97/17314 (WO 98/15556); International Patent Application No. PCT/US97/15899 (WO 98/11092); International Patent Application No. PCT/US97/15900 (WO 98/11096); International Patent Application No. PCT/US97/15801 (WO 98/11106); International Patent Application No. PCT/US97/15902 (WO 98/11097); International Patent Application No. PCT/US97/15903 (WO 98/11098); International Patent Application No. PCT/US97/15904; International Patent Application No. PCT/US97/15905 (WO 98/11099); International Patent Application No. PCT/US97/15906 (WO 98/11100); International Patent Application No. PCT/US97/15907 (WO 98/11093); International Patent Application No. PCT/US97/19976 (WO 98/11091); U.S. application Ser. No. 08/877049: U.S. application Ser. No. 08/877,366; U.S. application Ser. No. 08/877,399; U.S. application Ser. No. 08/877,336; U.S. application Ser. No. 08/877,269; U.S. application Ser. No. 08/877,050; U.S. application Ser. No. 08/877,052; U.S. application Ser. No. 08/877,051; U.S. application Ser. No. 08/877,498; U.S. application Ser. No. 08/877,057; U.S. application Ser. No. 08/877,739; U.S. application Ser. No. 08/877,677; U.S. application Ser. No. 08/877,741; U.S. application Ser. No. 08/877,743; U.S. application Ser. No. 08/877,457; U.S. application Ser. No. 08/877,673; U.S. application Ser. No. 08/876,507; and U.S. application Ser. No. 09/216,398 (each of which is herein incorporated by reference in its entirety).

Some FPT inhibitors are oligopeptides, especially tetrapeptides, or derivatives thereof, based on the formula Cys-Xaa1-Xaa2-Xaa3, where Xaa3 represents a serine, methionine or glutamine residue, and Xaa1 and Xaa2 can represent a wide variety of amino acid residues, but especially those with an aliphatic side-chain. Their derivatives can in some embodiments have three peptide bonds; thus it has been found that reduction of a peptide bond—CO—NH—to a secondary amine grouping, or even replacement of the nitrogen atoms in the peptide chain with carbon atoms (provided that certain factors such as general shape of the molecule and separation of the ends are largely conserved) affords compounds that are frequently more stable than the oligopeptides and, if active, have longer activity. Such compounds are referred to herein as peptido-mimetic compounds.

Oligopeptides (mostly tetrapeptides but also pentapeptides) including the formula Cys-Xaa1-Xaa2-Xaa3: EPA 461,489; EPA 520,823; EPA 528,486; and WO 95/11917, each of which is incorporated by reference in its entirety.

Peptido-mimetic compounds—especially Cys-Xaa-Xaa-Xaa-mimetics: EPA 535,730; EPA 535,731; EPA 618,221; WO 94/09766; WO 94/10138; WO 94/07966; U.S. Pat. No. 5,326,773; U.S. Pat. No. 5,340,828; U.S. Pat. No. 5,420,245; WO 95/20396; U.S. Pat. No. 5,439,918; and WO 95/20396 (each of which is herein incorporated by reference in its entirety).

Farnesylated peptido-mimetic compounds—specifically farnesylated Cys-Xaa-Xaa-Xaa-mimetic: GB-A 2,276,618 (herein incorporated by reference in its entirety).

Other peptido-mimetic compounds: U.S. Pat. No. 5,352,705; WO 94/00419; WO 95/00497; WO 95/09000; WO 95/09001; WO 95/12612; WO 95/25086; EPA 675,112; and FR-A 2,718,149 (each of which is herein incorporated by reference in its entirety).

Farnesyl derivatives: EPA 534,546; WO 94/19357; WO 95/08546; EPA 537,007; and WO 95/13059 (each of which is herein incorporated by reference in its entirety).

Natural products and derivatives: WO 94/18157; U.S. Pat. No. 5,430,055; GB-A 2,261,373; GB-A 2,261,374; GB-A 2,261,375; U.S. Pat. No. 5,420,334; U.S. Pat. No. 5,436,263 (each of which is herein incorporated by reference in its entirety).

Other compounds: WO 94/26723; WO95/08542; U.S. Pat. No. 5,420,157; WO 95/21815; WO 96/31501; WO 97/16443; WO 97/21701; U.S. Pat. No. 5,578,629; U.S. Pat. No. 5,627,202; WO 96/39137; WO 97/18813; WO 97/27752WO 97/27852; WO 97/27853; WO 97/27854; WO 97/36587; WO 97/36901; WO 97/36900; WO 97/36898; WO 97/36897; WO 97/36896; WO 97/36892; WO 97/36891; WO 97/36890; WO 97/36889; WO 97/36888; WO 97/36886; WO 97/36881; WO 97/36879; WO 97/36877; WO 97/36876; WO 97/36875; WO 97/36605; WO 97/36593; WO 97/36592; WO 97/36591; WO 97/36585; WO 97/36584; and WO 97/36583 (each of which is herein incorporated by reference in its entirety)

A plasmid encoding an α- and a β-unit of an FPT, and describing an assay therefor: WO 94/10184 (herein incorporated by reference in its entirety).

Reference is also made to U.S. application Ser. No. 09/217,335 and International Patent Application No. PCT/US98/26224, that disclose a variety of methods for combining FPT inhibitors with chemotherapeutic agents and/or radiation therapy in the treatment of proliferative disease such as cancer (both of which are herein incorporated by reference in their entirety).

All of the foregoing documents directed to compounds that are FPT inhibitors are incorporated herein by reference thereto.

Graham, in Exp. Opin. Ther. Patents (1995) 5(12): 1269-1285, gives a review of many such compounds (herein incorporated by reference in its entirety).

Indirect inhibition of farnesyl-protein transferase in vivo has been demonstrated with lovastatin (Merck & Co., Rahway, N.J.) and compactin (Hancock et al., ibid; Casey et al., ibid [NOTE TO INVENTORS: DO YOU HAVE THE FULL CITES FOR THESE?]; Schafer et al., Science 245:379 (1989), each of which is herein incorporated by reference in its entirety). These drugs inhibit HMG-CoA reductase, the rate limiting enzyme for the production of polyisoprenoids including farnesyl pyrophosphate. Inhibition of farnesyl pyrophosphate biosynthesis by inhibiting HMG-CoA reductase blocks Ras membrane localization in cultured cells.

Another inhibitor of Rho is S-farnesylthiosalicylic acid (FTS) and its derivatives and analogs. Another inhibitor is imidazole-containing benzodiazepines and analogs (WO-97/30992). The Rho inhibitor can also act downstream by interaction with ROCK (Rho activated kinase) leading to an inhibition of Rho. Such inhibitors are described in U.S. Pat. No. 6,642,263 (each of which is herein incorporated by reference in its entirety).

Other Rho inhibitors that may be used are described in U.S. Pat. Nos. 6,642,263, 6,451,825. Such inhibitors can be found using conventions cell screening assays, e.g., described in U.S. Pat. No. 6,620,591 (all of which are herein incorporated by reference in their entirety).

In another embodiment, compounds that are non-selective FPTase/GGTase inhibitors can be used. (Nagasu et al. Cancer Research, 55:5310-5314 (1995); PCT application WO 95/25086, each of which is herein incorporated by reference in its entirety). Recently, synergy between certain modulating anions and farnesyl-diphosphate competitive inhibitors of FPTase has been disclosed (J. D. Scholten et al. J. Biol. Chem. 272:18077-18081 (1997) incorporated by reference in its entirety). Particular examples of modulating anions useful in the instant GGTase-I inhibition assay include adenosine 5′-triphosphate (ATP), 2′-deoxyadenosine 5′-triphosphate (dATP), 2′-deoxycytosine 5′-triphosphate (dCTP), beta-glycerol phosphate, pyrophosphate, guanosine 5′-triphosphate (GTP), 2′-deoxyguanosine 5′-triphosphate (dGTP), uridine 5′-triphosphate, dithiophosphate, 3′-deoxythymidine 5′-triphosphate, tripolyphosphate, D-myo-inositol 1,4,5-triphosphate, chloride, guanosine 5′-monophosphate, 2′-deoxyguanosine 5′-monophosphate, orthophosphate, formycin A, inosine diphosphate, trimetaphosphate, sulfate and the like. Preferably, the modulating anion is selected from adenosine 5′-triphosphate, 2′-deoxyadenosine 5′-triphosphate, 2′-deoxycytosine 5′-triphosphate, beta-glycerol phosphate, pyrophosphate, guanosine 5′-triphosphate, 2′-deoxyguanosine 5′-triphosphate, uridine 5′-triphosphate, dithiophosphate, 3′-deoxythymidine 5′-triphosphate, tripolyphosphate, D-myo-inositol 1,4,5-triphosphate and sulfate. Most preferably, the modulating anion is selected from adenosine 5′-triphosphate, β-glycerol phosphate, pyrophosphate, dithiophosphate and sulfate.

Inhibitors of geranylgeranyl-protein transferase (GGT) have been described in U.S. Pat. No. 5,470,832 (Gibbs & Graham) (incorporated by reference in its entirety). These compounds can be administered to an individual in dosage amounts of between 0.5 mg/kg of body weight to about 20 mg/kg of body weight. Alternatively, one or more inhibitors of isoprenylation, including farnesyl transferase (FT) inhibitors and/or geranylgeranyl transferase inhibitors (GGT) are administered to a patient.

### Toxins

Toxins can also be used such as Toxins A and B from C. difficile and C. sordellii lethal toxin (LT). In addition, RhoA, RhoB and/or RhoC can be inhibited when Rho is specifically ADP ribosylated by C3 enzyme, that is one of the botulinum toxins, and Staphylococcal toxin EDIN (Narumiya, S. and Morii, S., Cell Signal, 5, 9-19, 1993; Sekine, A. et al., J. Biol. Chem., 264, 8602-8605, 1989, each of which is incorporated by reference in its entirety). The term C3 refers to C3 ADP-ribosyltransferase, a specific Rho inactivator. A preferred representative example is C3 ADP-ribosyltransferase, a 23 KDa exoenzyme secreted from certain strains of types C and D from Clostridium botulinum, that specifically ADP-ribosylates the Rho family of these GTP-binding proteins.

### Antisense

The present methods can employ antisense compounds, particularly oligonucleotides, for use in modulating the function of nucleic acid molecules encoding Rho, including but not limited to, RhoA, RhoB and RhoC, ultimately modulating the amount of Rho produced. This is accomplished by providing oligonucleotides that specifically hybridize with nucleic acids, preferably mRNA, encoding for RhoA.

The term “antisense”, as used herein, refers to nucleotide sequences that are complementary to a specific DNA or RNA sequence. The term “antisense strand” is used in reference to a nucleic acid strand that is complementary to the “sense” strand. Antisense molecules can be produced by any method, including synthesis by ligating the gene(s) of interest in a reverse orientation to a viral promoter, that permits the synthesis of a complementary strand. Once introduced into a cell, this transcribed strand combines with natural sequences produced by the cell to form duplexes. These duplexes then block either the further transcription or translation. In this manner, mutant phenotypes can be generated. The designation “negative” is sometimes used in reference to the antisense strand, and “positive” is sometimes used in reference to the sense strand.

HMG-CoA Reductase

HMG-CoA (3-hydroxy-3-methylglutaryl-coenzyme A) reductase is the microsomal enzyme that catalyzes the rate limiting reaction in cholesterol biosynthesis (HMG-CoA6Mevalonate). An HMG-CoA reductase inhibitor inhibits HMG-CoA reductase.

HMG-CoA reductase inhibitors useful as modulators include, but are not limited to, simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. Nos. 5,622,985, 5,135,935, 5,356,896, 4,920,109, 5,286,895, 5,262,435, 5,260,332, 5,317,031, 5,283,256, 5,256,689, 5,182,298, 5,369,125, 5,302,604, 5,166,171, 5,202,327, 5,276,021, 5,196,440, 5,091,386, 5,091,378, 4,904,646, 5,385,932, 5,250,435, 5,132,312, 5,130,306, 5,116,870, 5,112,857, 5,102,911, 5,098,931, 5,081,136, 5,025,000, 5,021,453, 5,017,716, 5,001,144, 5,001,128, 4,997,837, 4,996,234, 4,994,494, 4,992,429, 4,970,231, 4,968,693, 4,963,538, 4,957,940, 4,950,675, 4,946,864, 4,946,860, 4,940,800, 4,940,727, 4,939,143, 4,929,620, 4,923,861, 4,906,657, 4,906,624 and 4,897,402, the disclosures of which are incorporated herein by reference.

In preferred embodiments, the HMG-CoA reductase inhibitor is administered in an amount of about 10 milligrams per day to about 300 milligrams per day; preferably in an amount of about 20 milligrams per day to about 200 milligrams per day; and the isosorbide mononitrate is administered in an amount of about 30 milligrams per day to about 150 milligrams per day. The preferred amounts of HMG-CoA reductase inhibitor can be administered as a single dose once a day; in multiple doses several times throughout the day; or in a sustained-release formulation.

Preferably, the HMG-CoA reductase inhibitor is a statin, including, but not limited to, lovastatin (MEVACOR), simvastatin (ZOCOR), pravastatin (PRAVACHOL), fluvastatin, cerivastatin (BAYCOL), atorvastatin (LIPITOR), and the like. In some embodiments, the Rho inhibitor is selected from the group consisting of: (5S,22S)-19,20-dihydro-3-methyl-19-oxo-5H-5,22: 18,21-diethano-12,14-etheno-6, 10-metheno-22H-benzo[d]imidazo[4,3-k][1,6,9, 12]oxatriazacyclooctadecine-9-carbonitrile; (5R,22R)-19,20-dihydro-3-methyl-19-oxo-5H-5,22: 18,21-diethano-12,14-etheno-6,10-metheno-22H-benzo[d]imidazo[4,3-k][1,6,9, 12]oxatriazacyclooctadecine-9-carbonitrile; (5S,22R)-19,20-dihydro-3-methyl-19-oxo-5H-5,22: 18,21-diethano-12,14-etheno-6,10-metheno-22H-benzo[d]imidazo[4,3-k][1,6,9, 12]oxatriazacyclooctadecine-9-carbonitrile; (5R,22S)-19,20-dihydro-3-methyl-19-oxo-5H-5,22: 18,21-diethano-12,14-etheno-6,10-metheno-22H-benzo[d]imidazo[4,3-k][1,6,9, 12]oxatriazacyclooctadecine-9-carbonitrile; (+/−)-(5R*,22R*)-19,20-dihydro-3-methyl-19-oxo-5H-5,22: 18,21-diethano-12,14-etheno-6,10-metheno-22H-benzo[d]imidazo[4,3-k][1,6,9, 12]oxatriazacyclooctadecine-9-carbonitrile; and(+/−)-(5R*,22S*)-19,20-dihydro-3-methyl-19-oxo-5H-5,22: 18,21-diethano-12,14-etheno-6,10-metheno-22H-benzo[d]imidazo[4,3-k][1,6,9, 12]oxatriazacyclooctadecine-9-carbonitrile, or a pharmaceutically acceptable salt thereof.

### Small Molecule Inhibitors

Small molecule inhibitors can be used to inhibit and/or modulate the GTPases as disclosed herein. Any type of small molecule inhibitor which is known to one of skill in the art may be used, including but not limited to, those disclosed in U.S. patent application Ser. No.______, filed Nov. 20, 2004, entitled “GTPase Inhibitors and Methods of Use”, attorney docket number CHMC24.001A, based on Provisional application 60/523,599 entitled “GTPase inhibitors and methods of Use”, filed Nov. 20, 2003, each of which is herein incorporated by reference in its entirety. Many methods are known to identify small molecule inhibitors and commercial laboratories are available to screen for small molecule inhibitors. In addition to the method disclosed in Provisional patent application 60/523,599 entitled “GTPase Inhibitors and methods of Use”, robotic screening assays are available which look at the ability of small molecules to inhibit the GTPase activity of choice in an appropriate assay. For example, chemicals can be obtained from the compound collection at Merck Research Laboratories (Rahway, N.J.) or a like company. The compounds can be screened for inhibition of a specific GTPase by automated robotic screening in a 96-well plate format. In summary, the compounds can be dissolved at an initial concentration of about 50 μM in DMSO and dispensed into the 96-well plate. The 96-well plate assay may contain an appropriate number of units of the GTPase of choice and a substrate. Compounds that cause greater than a 50% inhibition of GTPase activity can be further diluted and tested to establish the concentration necessary for a 50% inhibition of activity.

The Rho family Guanosine Triphosphatases (GTPases), Rac1 and Rac2, are important signaling regulators in hematopoietic cells. Inhibition of both Rac1 and Rac2 alleles leads to massive mobilization of hematopoietic stem/progenitor cells (HSC/P) whereas inhibition of Rac1, but not Rac2, HSC/P fail to reconstitute irradiated recipient mice. In HSC/P, Rac1 controls proliferation via p42/p44 MAPKs, Cyclin D1 and p27. In contrast, in neutrophils Rac2, but not Rac1, regulates superoxide production and cell migration. In both cell types, each GTPase plays a distinct role in organizing actin. Thus, inhibition of Rac1 and Rac2 is allows for the mobilization of hematopoietic cells.

### Chimeric Inhibitors

In another embodiment, any chimeric peptides cam be used as inhibitors/modulators known to one of skill in the art, including but not limited to those disclosed in related U.S. patent application Ser. No.______, filed on Aug. 13, 2004 entitled “Chimeric Peptides for the Regulation of GTPases” (attorney docket number CHMC21.001A), based on Provisional application 60/494,719, entitled, “Chimeric Peptides for the Regulation of GTPases”, filed Aug. 13, 2003, each of which is herein incorporated by reference in its entirety.

### Polypeptide Inhibitors

A polypeptide can be produced in an expression system, e.g., in vivo, in vitro, cell-free, recombinant, cell fusion, etc. Modifications to the polypeptide imparted by such system include, glycosylation, amino acid substitution (e.g., by differing codon usage), polypeptide processing such as digestion, cleavage, endopeptidase or exopeptidase activity, attachment of chemical moieties, including lipids, phosphates, etc. For example, some cell lines can remove the terminal methionine from an expressed polypeptide.

A polypeptide can be recovered from natural sources, transformed host cells (culture medium or cells) according to the usual methods, including, ammonium sulfate or ethanol precipitation, acid extraction, anion or cation exchange chromatography, phosphocellulose chromatography, hydrophobic interaction chromatography, hydroxyapatite chromatography and lectin chromatography. It may be useful to have low concentrations (approximately 0.1-5 mM) of calcium ion present during purification (Price, et al., J. Biol. Chem., 244:917 (1969) incorporated by reference in its entirety). Protein refolding steps can be used, as necessary, in completing the configuration of the mature protein. Finally, high performance liquid chromatography (HPLC) can be employed for final purification steps. A nucleic acid comprising a nucleotide sequence coding for a polypeptide disclosed herein can include only coding sequence of chimeric GAP; coding sequence of chimeric GAP and additional coding sequence (e.g., sequences coding for leader, secretory, targeting, enzymatic, fluorescent or other diagnostic peptides), coding sequence of chimeric GAP and non-coding sequences, e.g., untranslated sequences at either a 5′ or 3′ end, or dispersed in the coding sequence, e.g., introns. A nucleic acid comprising a nucleotide sequence coding without interruption for a chimeric GAP polypeptide means that the nucleotide sequence contains an amino acid coding sequence for a chimeric GAP polypeptide, with no non-coding nucleotides interrupting or intervening in the coding sequence, e.g., absent intron(s). Such a nucleotide sequence can also be described as contiguous.

### Nucleic Acids

Nucleic acids as disclosed in certain embodiments herein can comprise an expression control sequence operably linked to a nucleic acid as described above. The phrase “expression control sequence” preferably means a nucleic acid sequence that regulates expression of a polypeptide coded for by a nucleic acid to which it is operably linked. Expression can be regulated at the level of the mRNA or polypeptide. Thus, the expression control sequence can include mRNA-related elements and protein-related elements. Such elements can include promoters, enhancers (viral or cellular), ribosome binding sequences, transcriptional terminators, etc. An expression control sequence is preferably operably linked to a nucleotide coding sequence when the expression control sequence is positioned in such a manner to effect or achieve expression of the coding sequence. For example, when a promoter is operably linked 5′ to a coding sequence, the promoter can drive expression of the coding sequence. Expression control sequences can be heterologous or endogenous to the normal gene.

Nucleic acids as disclosed herein can be selected on the basis of nucleic acid hybridization. The ability of two single-stranded nucleic acid preparations to hybridize together is a measure of their nucleotide sequence complementarity, e.g., base-pairing between nucleotides, such as A-T, G-C, etc.

A nucleic acid or polypeptide can comprise one or more differences in the nucleotide or amino acid sequence. Changes or modifications to the nucleotide and/or amino acid sequence can be accomplished by any method available, including directed or random mutagenesis. A nucleotide sequence coding for a chimeric GAP polypeptide can contain codons found in a naturally-occurring gene, transcript, or cDNA or it can contain degenerate codons coding for the same amino acid sequences.

A nucleic acid can comprise, e.g., DNA, RNA, synthetic nucleic acid, peptide nucleic acid, modified nucleotides, or mixtures. A DNA can be double- or single-stranded. Nucleotides comprised in a nucleic acid can be joined via various known linkages, e.g., ester, sulfamate, sulfamide, phosphorothioate, phosphoramidate, methylphosphonate, carbamate, etc., depending on the desired purpose, e.g., resistance to nucleases, such as RNase H, improved in vivo stability, etc. See, e.g., U.S. Pat. No. 5,378,825 (herein incorporated by reference in its entirety).

### Pharmaceutical Compositions

The active compounds can be incorporated into pharmaceutical compositions suitable for administration to a subject, for example, a human. Such compositions typically comprise the nucleic acid molecule, protein, modulator, or antibody and a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions. Supplementary active compounds can also be incorporated into the compositions. A pharmaceutical composition is formulated to be compatible with its intended route of administration. Examples of routes of administration include parenteral, including but not limited to, intravenous, intradermal, subcutaneous, oral (for example, inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid, buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc., liposomal suspensions (including liposomes targeted to infected cells with monoclonal antibodies to viral antigens) can also be used as pharmaceutically acceptable carriers. These can be prepared according to methods known to those skilled in the art.

It is especially advantageous to formulate oral or parenteral compositions in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms are dictated by and directly dependent on the unique characteristics of the active compound and the particular therapeutic effect to be achieved, and the limitations inherent in the art of compounding such an active compound for the treatment of individuals.

In practicing the method of treatment or use of the present methods and compounds, a therapeutically effective amount of one, two, or more of the synthetic oligonucleotides is administered to a subject afflicted with a disease or disorder related to Rho family GTPases, or to a tissue that has such disease or disorder. The active agents can be administered in accordance with the method either alone or in combination with other known therapies. When co-administered with one or more other therapies, the active agents can be administered either simultaneously with the other treatment(s), or sequentially. If administered sequentially, the attending physician will decide on the appropriate sequence of administering the active agents in combination with the other therapy.

Generally, a therapeutically effective amount of active agent (i.e., an effective dosage) ranges from about 0.001 to 5000 mg/kg body weight, more preferably about 0.01 to 1000 mg/kg body weight, more preferably about 0.01 to 500 mg/kg body weight, more preferably about 0.01 to 250 mg/kg body weight, more preferably about 0.01 to 100 mg/kg body weight, more preferably about 0.001 to 60 mg/kg body weight, more preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount can include a single treatment or, preferably, can include a series of treatments. In a preferred example, a subject is treated in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage used for treatment can increase or decrease over the course of a particular treatment. Changes in dosage can result and become apparent from the results of diagnostic assays as described herein.

### Additional Rho GTPase Modulators

One or more additional agents are encompassed by various embodiments that modulate expression or activity of Rho GTPase. An agent can, for example, be a small molecule. For example, such small molecules include, but are not limited to, peptides, peptidomimetics, amino acids, and amino acid analogs.

### Administration

The compounds and compositions can be administered by any available and effective delivery system including, but not limited to, orally, bucally, parenterally, by inhalation spray, topically (including transdermally), or rectally in dosage unit formulations containing conventional nontoxic pharmaceutically acceptable carriers, adjuvants, and vehicles as desired. The preferred methods of administration are by oral administration or topical application (transdermal application).

Topical administration can also involve the use of transdermal administration such as transdermal patches or iontophoresis devices. Dosage forms for topical administration of the compounds and compositions can include creams, sprays, lotions, gels, ointments, and the like. In such dosage forms, the compositions can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application.

The compositions can also be applied topically using a transdermal system, such as one of an acrylic-based polymer adhesive with a resinous crosslinking agent impregnated with the composition and laminated to an impermeable backing. In a preferred embodiment, the compositions are administered in the form of a transdermal patch, more preferably in the form of a sustained-release transdermal patch. The transdermal patches can include any conventional form such as, for example, adhesive matrix, polymeric matrix, reservoir patch, matrix or monolithic-type laminated structure, and are generally comprised of one or more backing layers, adhesives, penetration enhancers, an optional rate controlling membrane and a release liner that is removed to expose the adhesives prior to application. Polymeric matrix patches also comprise a polymeric-matrix forming material. Suitable transdermal patches are described in more detail in, for example, U.S. Pat. Nos. 5,262,165, 5,948,433, 6,010,715 and 6,071,531, the disclosure of each of which are incorporated herein by reference in its entirety.

Solid dosage forms for oral administration can include capsules, sustained-release capsules, tablets, chewable tablets, sublingual tablets, effervescent tablets, pills, powders, granules and gels. In such solid dosage forms, the active compounds can be admixed with at least one inert diluent, such as sucrose, lactose or starch. Such dosage forms can also comprise, as is normal practice, additional substances other than inert diluents, e.g., lubricating agents, such as magnesium stearate. In the case of capsules, tablets, effervescent tablets, and pills, the dosage forms can also comprise buffering agents. Soft gelatin capsules can be prepared to contain a mixture of the active compound or composition and vegetable oil. Hard gelatin capsules can contain granules of the active compound in combination with a solid, pulverulent carrier, such as lactose, saccharose, sorbitol, mannitol, potato starch, corn starch, amylopectin, or cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents. Suppositories for rectal administration of the compounds or compositions can be prepared by mixing the drug with a suitable nonirritating excipient such as cocoa butter and polyethylene glycols that are solid at room temperature but liquid at rectal temperature, such that they will melt in the rectum and release the drug.

The term parenteral includes subcutaneous injections, intravenous, intramuscular, intrasternal injection, or infusion techniques. Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compounds and compositions are typically be administered in a pharmaceutical composition comprising one or more carriers or excipients.

Examples of suitable carriers include, for example, water, silicone, waxes, petroleum jelly, polyethylene glycols, propylene glycols, liposomes, sugars, salt solutions, alcohol, vegetable oils, gelatins, lactose, amylose, magnesium stearate, talc, surfactants, silicic acids, viscous paraffins, perfume oils, fatty acid monoglycerides and diglycerides, petroethral fatty acid esters, hydroxymethylcelluloses, polyvinyl-pyrrolidones, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavorings and/or aromatic substances and the like that do not deleteriously react with the active compounds. For topical application, the compositions can also include one or more permeation enhancers including, for example, dimethylsulfoxide (DMSO), dimethyl formamide (DMF), N,N-dimethylacetamide (DMA), decylmethylsulfoxide (C10MSO), polyethylene glycol monolaurate (PEGML), glyceral monolaurate, lecithin, 1-substituted azacycloheptan-2-ones, particularly 1-N-dodecylcyclazacycoheptan-2-ones (available under the trademark AZONE from Nelson Research & Development Co., Irvine Calif.), alcohols and the like. For parenteral application, particularly suitable vehicles consist of solutions, preferably oily or aqueous solutions, as well as suspensions, emulsions, or implants. Aqueous suspensions can contain substances that increase the viscosity of the suspension and include, for example, sodium carboxymethyl cellulose, sorbitol and/or dextran. Optionally, the suspension can contain stabilizers. The compositions, if desired, can also contain minor amounts of wetting agents, emulsifying agents and/or pH buffering agents.

Various delivery systems are known and can be used to administer the compounds or compositions, including, for example, encapsulation in liposomes, microbubbles, emulsions, microparticles, microcapsules and the like. The required dosage can be administered as a single unit or in a sustained release form.

While individual needs can vary, determination of optimal ranges for effective amounts of the compounds and/or compositions is within the skill of the art and can be determined by standard clinical techniques, including reference to Goodman and Gilman, supra; The Physician's Desk Reference, Medical Economics Company, Inc., Oradell, N.J., 1995; and Drug Facts and Comparisons, Inc., St. Louis, Mo., 1993. Generally, the dosage required to provide an effective amount of the compounds and compositions, that can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The compounds and compositions can be formulated as pharmaceutically acceptable salts. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts can be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like.

The pharmaceutically acceptable salts of the compounds can be synthesized from the compounds that contain a basic moiety by conventional chemical methods. Generally, the salts are prepared either by ion exchange chromatography or by reacting the free base with stoichiometric amounts or with an excess of the desired salt-forming inorganic or organic acid in a suitable solvent or various combinations of solvents.

### Exemplary Uses

The data described herein demonstrates that manipulations of Rho activity can be used for therapeutic purposes to influence self renewal and differentiation of hematopoietic stem cells, and to replace damaged or defective cells. Areas of application include, but are not limited to, enhancement of blood cell and myeloid cell formation following high dose chemotherapy in cancer treatment; improved engraftment following bone marrow or stem cell transplantations, and gene therapy; stem cell therapy by amplifying the undifferentiated cells of erythroid and myeloid lineages and applying appropriate factors to induce terminal differentiation; enhancing engraftment after infusion into a recipient, and regulation of formation of various blood cell components for treating hematological and autoimmune disorders.

Some embodiments of the invention, therefore, are directed to methods to elevate the progenitor cells and/or stem cells, in a subject, which method comprises administering to said subject an amount of an active compound effective to elevate progenitor cell and/or stem cell levels is disclosed herein.

In some embodiments, hematopoietic progenitor and/or stem cells are engrafted for gene therapy. As used herein, “gene therapy” or a “genetic modification” refers to any addition, deletion or disruption to a cell's normal nucleotides. The methods are intended to encompass any method of gene transfer into hematopoietic stem cells, including but not limited to viral mediated gene transfer, liposome mediated transfer, transformation, transfection and transduction, e.g., viral mediated gene transfer such as the use of vectors based on DNA viruses such as adenovirus, adeno-associated virus and herpes virus, as well as retroviral based vectors. A “viral vector” is defined as a recombinantly produced virus or viral particle that comprises a polynucleotide to be delivered into a host cell, either in vivo, ex vivo or in vitro. Examples of viral vectors include retroviral vectors such as lentiviral vectors; adenovirus vectors; adeno-associated virus vectors and the like. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” can carry the same meaning and generally refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism. Retroviruses carry their genetic information in the form of RNA; however, once the virus infects a cell, the RNA is reverse-transcribed into the DNA form, that integrates into the genomic DNA of the infected cell. The integrated DNA form is called a provirus.

In aspects where gene transfer is mediated by a DNA viral vector, such as a adenovirus (Ad) or adeno-associated virus (AAV), a vector construct refers to the polynucleotide comprising the retroviral genome or part thereof, and a therapeutic gene. Adenoviruses (Ads) are a relatively well characterized, homogenous group of viruses, including over 50 serotypes. Adeno-associated virus (AAV) has also been used as a gene transfer system. (See, e.g., U.S. Pat. Nos. 5,693,531 and 5,691,176, both of which is incorporated by reference in its entirety).

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that can interfere with or reduce expression, either at the level of transcription or translation. Alternatively, consensus ribosome binding sites can be inserted immediately 5′ of the start codon to enhance expression. Examples of vectors are viruses, such as baculovirus and retrovirus, bacteriophage, cosmid, plasmid, fungal vectors and other recombination vehicles typically used in the art that have been described for expression in a variety of eukaryotic and prokaryotic hosts, and can be used for gene therapy as well as for simple protein expression.

Among these are several non-viral vectors, including DNA/liposome complexes, and targeted viral protein DNA complexes. To enhance delivery to a cell, the nucleic acid or proteins can be conjugated to antibodies or binding fragments thereof that bind cell surface antigens, e.g., TCR, CD3 or CD4. Liposomes that also comprise a targeting antibody or fragment thereof can be used in the methods. Targeting complexes for use in the methods disclosed herein are also provided.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Other means are well known and available in the art.

The methods are intended to encompass any method of gene transfer into hematopoietic stem cells, including but not limited to viral mediated gene transfer, liposome mediated transfer, transformation, transfection and transduction, e.g., viral mediated gene transfer such as the use of vectors based on DNA viruses such as adenovirus, adeno-associated virus and herpes virus, as well as retroviral based vectors. The methods are particularly suited for the integration of a nucleic acid contained in a vector or construct lacking a nuclear localizing element or sequence such that the nucleic acid remains in the cytoplasm. In these instances, the nucleic acid or therapeutic gene is able to enter the nucleus during M (mitosis) phase when the nuclear membrane breaks down and the nucleic acid or therapeutic gene gains access to the host cell chromosome. In some embodiments, nucleic acid vectors and constructs having a nuclear localizing element or sequence are specifically excluded.

The HSC cultures described herein are particularly suited for retroviral mediated gene transfer. In retroviral transduction, the transferred sequences are stably integrated into the chromosomal DNA of the target cell. Conditions that favor stable proviral integration include actively cycling cells, as provided for herein.

The HSC cells are transduced with a therapeutic gene. Preferably, the transduction is via a vector such as a retroviral vector. When transduction is ex vivo, the transduced cells are subsequently administered to the recipient. Thus, treatment of diseases amenable to gene transfer into HSCs are encompassed by administering the gene ex vivo or in vivo by the methods disclosed herein. For example, diseases including, but not limited to, beta-thalassemia, sickle cell anemia, adenosine deaminase deficiency, recombinase deficiency, recombinase regulatory gene deficiency, etc. can be corrected by introduction of a therapeutic gene. Other indications of gene therapy are introduction of drug resistance genes to enable normal stem cells to have an advantage and be subject to selective pressure during chemotherapy. Suitable drug resistance genes include, but are not limited to, the gene encoding the multidrug resistance (MDR) protein.

Diseases other than those associated with hematopoietic cells can also be treated by genetic modification, where the disease is related to the lack of a particular secreted product including, but not limited to, hormones, enzymes, interferons, growth factors, or the like. By employing an appropriate regulatory initiation region, inducible production of the deficient protein can be achieved, so that production of the protein will parallel natural production, even though production will be in a different cell type from the cell type that normally produces such protein. It is also possible to insert a ribozyme, antisense or other message to inhibit particular gene products or susceptibility to diseases, particularly hematolymphotropic diseases.

Retroviral vectors useful in the methods are produced recombinantly by procedures already taught in the art. As is apparent to the skilled artisan, the retroviral vectors useful in the methods are capable of infecting HSCs. The techniques used to construct vectors, and transfect and infect cells are widely practiced in the art. Examples of retroviral vectors are those derived from murine, avian or primate retroviruses.

In producing retroviral vector constructs derived from the Moloney murine leukemia virus (MoMLV), in most cases, the viral gag, pol and env sequences are removed from the virus, creating room for insertion of foreign DNA sequences. Genes encoded by the foreign DNA are usually expressed under the control of the strong viral promoter in the LTR. Such a construct can be packed into viral particles efficiently if the gag, pol and env functions are provided in trans by a packaging cell line. Thus, when the vector construct is introduced into the packaging cell, the gag-pol and env proteins produced by the cell, assemble with the vector RNA to produce infectious virions that are secreted into the culture medium. The virus thus produced can infect and integrate into the DNA of the target cell, but does not produce infectious viral particles since it is lacking essential packaging sequences. Most of the packaging cell lines currently in use have been transfected with separate plasmids, each containing one of the necessary coding sequences, so that multiple recombination events are necessary before a replication competent virus can be produced. Alternatively, the packaging cell line harbors an integrated provirus.

The range of host cells that can be infected by a retrovirus or retroviral vector is determined by the viral envelope protein. The recombinant virus can be used to infect virtually any other cell type recognized by the env protein provided by the packaging cell, resulting in the integration of the viral genome in the transduced cell and the stable production of the foreign gene product. In general, murine ecotropic env of MoMLV allows infection of rodent cells, whereas amphotropic env allows infection of rodent, avian and some primate cells, including human cells.

Usually, the vectors will contain at least two heterologous genes or gene sequences: (i) the therapeutic gene to be transferred; and (ii) a marker gene that enables tracking of infected cells. As used herein, “therapeutic gene” can be an entire gene or only the functionally active fragment of the gene capable of compensating for the deficiency in the patient that arises from the defective endogenous gene. Therapeutic gene also encompasses antisense oligonucleotides or genes useful for antisense suppression and ribozymes for ribozyme-mediated therapy. Therapeutic genes that encode dominant inhibitory oligonucleotides and peptides as well as genes that encode regulatory proteins and oligonucleotides also are encompassed. Generally, gene therapy will involve the transfer of a single therapeutic gene although more than one gene can be used for the treatment of particular diseases.

Nucleotide sequences for the therapeutic gene will generally be known in the art or can be obtained from various sequence databases such as GenBank. The therapeutic gene itself will generally be available or can be isolated and cloned using the polymerase chain reaction PCR (Perkin-Elmer) and other standard recombinant techniques. The skilled artisan will readily recognize that any therapeutic gene can be excised as a compatible restriction fragment and placed in a vector in such a manner as to allow proper expression of the therapeutic gene in hematopoietic cells.

The viral constructs can be prepared in a variety of conventional ways. Numerous vectors are now available that provide the desired features, such as long terminal repeats, marker genes, and restriction sites, that can be further modified by techniques known in the art. Preferably, the foreign gene(s) is under the control of a cell specific promoter.

Examples of promoters that can be used to cause expression of the introduced sequence in specific cell types include Granzyme A for expression in T-cells and NK cells, the CD34 promoter for expression in stem and progenitor cells, the CD8 promoter for expression in cytotoxic T-cells, and the CD11b promoter for expression in myeloid cells. Inducible promoters can be used for gene expression under certain physiologic conditions.

### Engraftment of Blood Precursors

Methods are provided to enhance or facilitate hematopoietic reconstitution or engraftment, in mammals, including humans. “Peripheral blood precursor cells”, as used herein, include stem cells, that are pluripotent, and early progenitor cells, that are more differentiated but have a greater potential for proliferation than stem cells. In some embodiments, engraftment of peripheral blood precursor cells in a mammal is induced by administering to the mammal an effective amount of an active compound.

The methods can additionally be used for gene therapy. Because pluripotent stem cells are self-renewing, and give rise to cell progenitors as well as mature blood cells, the stem cells are an appropriate target for gene therapy. After mobilization, stem cells can be collected. The stem cells can be modified to deliver gene products upon reintroduction to the individual. After modification, the cells are reinfused into the affected individual. Engraftment of the reinfused cells can then be facilitated by use of the Rho modulator prior to, concurrently or subsequent to infusion of cells.

As used herein, the term “progenitor cells” refers to cells that, in response to certain stimuli, can form differentiated hematopoietic or myeloid cells. The presence of progenitor cells can be assessed by the ability of the cells in a sample to form colony-forming units of various types, including, for example, CFU-GM (colony-forming units, granulocyte-macrophage); CFU-GEMM (colony-forming units, multipotential); BFU-E (burst-forming units, erythroid); HPP—CFC (high proliferative potential colony-forming cells); or other types of differentiated colonies that can be obtained in culture using known protocols. As used herein, “stem” cells are less differentiated forms of progenitor cells. Typically, such cells are often positive for CD34 in humans. Some stem cells do not contain this marker, however.

Typical conditions that can be ameliorated or otherwise benefited by the method include hematopoietic disorders, such as aplastic anemia, leukemias, drug-induced anemias, genetic disorders and hematopoietic deficits from chemotherapy or radiation therapy.

Suitable dosage ranges for the active compound vary according to these considerations, but in general, the compounds are administered in the range of about 0.1 μg/kg-5 mg/kg of body weight; preferably the range is about 1 μg/kg-300 μg/kg of body weight; more preferably about 10 μg/kg-100 μg/kg of body weight. For a typical 70-kg human subject, thus, the dosage range is from about 0.7 μg-350 mg; preferably about 700 μg-21 mg; most preferably about 700 μg-7 mg. Dosages can be higher when the compounds are administered orally or transdermally as compared to, for example, i.v. administration. The compounds can be administered as a single bolus dose, a dose over time, as in i.v. or transdermal administration, or in multiple dosages.

The amount of active compound to be administered can vary according to the discretion of the skilled artisan. The amount of active compound to be administered to the recipient is within the ranges described above for stem cell engraftment. However, the administration of such amounts will vary according to the standards set forth by clinicians in the field of stem cell enhancement therapy. Administration should generally occur daily following chemotherapy or other treatment for 1 or more days, preferably daily or intermittently for up to 200 days.

The dosage regimen for engrafting hematopoietic progenitor cells from bone marrow into peripheral blood with the active compounds is based on a variety of factors, including the type of injury, the age, weight, sex, medical condition of the individual, the severity of the condition, the route of administration, and the particular compound employed. Thus, the dosage regimen can vary widely, but can be determined routinely by a physician using standard methods. Dosage levels of the order of between 0.1 ng/kg and 10 mg/kg body weight of the active compounds per body weight are useful for all methods of use disclosed herein.

The treatment regime will also vary depending on the disease being treated, based on a variety of factors, including the type of injury, the age, weight, sex, medical condition of the individual, the severity of the condition, the route of administration, and the particular compound employed. For example, the active compounds are administered to an oncology patient for up to 30 days prior to a course of chemotherapy and for up to 60 days post-chemotherapy. The therapy is administered for 1 to 6 times per day at dosages as described above.

In a preferred embodiment, the active compound is administered subcutaneously. A suitable subcutaneous dose of the active compound is preferably between about 0.1 ng/kg and about 10 mg/kg administered twice daily for a time sufficient to increase engraftment of hematopoietic stem and progenitor cells from bone marrow into peripheral blood. This dosage regimen maximizes the therapeutic benefits of the methods and compositions while minimizing the amount of agonist needed. Such an application minimizes costs as well as possible deleterious side effects.

For subcutaneous administration, the active ingredient can comprise from 0.0001% to 10% w/w, e.g. from 1% to 2% by weight of the formulation, although it can comprise as much as 10% w/w, but preferably not more than 5% w/w, and more preferably from 0.1% to 1% of the formulation. In a most preferred embodiment, subcutaneous administration of between about 1 to 1000 μg/kg/day of the active compounds is initiated at between one week before to one week after administration of a chemotherapeutic agent.

In another preferred embodiment, a subject undergoes repeated cycles of treatment according to the method. Preferably, a subsequent treatment cycle commences only after the administration of the compounds have been terminated and the subject's blood cell counts (e.g., white blood cell count) have returned to a therapeutically acceptable level (as determined by the attending veterinarian or physician), permitting the repeated chemotherapy.

In all of these embodiments, the compounds can be administered prior to, simultaneously with, or subsequent to chemotherapeutic exposure.

The compounds may be administered as sole active ingredients and/or in admixture with additional active ingredients that are therapeutically or nutritionally useful, such as antibiotics, vitamins, herbal extracts, anti-inflammatories, glucose, antipyretics, analgesics, granulocyte-macrophage colony stimulating factor (GM-CSF), Interleukins (IL-1, IL-2, IL-3, IL-4, IL-5, IL-6, IL-7, IL-8, IL-9, IL-10 IL-11, IL-12, IL-13, IL-14, or IL-15), TPO, SCF, or other growth factor such as CSF-1, SF, EPO, leukemia inhibitory factor (LIF), or fibroblast growth factor (FGF), as well as C-KIT ligand, M-CSF and TNF-α, PIXY-321 (GM-CSF/IL-3 fusion protein), macrophage inflammatory protein, stem cell factor, thrombopoietin, growth related oncogene or chemotherapy and the like.

The term, “in conjunction with”, as used herein, refers to concurrent administration of the active compound with the growth factor, as well as administration of the active compound within several days (e.g., within approximately 1 to 7 days) of administration of the growth factor. Administration of the growth factor can be before or after administration of the active compound.

The active compounds can be administered by any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intraarterial, intramuscular, intrastemal, intratendinous, intraspinal, intracranial, intrathoracic, infusion techniques or intraperitoneally.

The active compounds can be made up in a solid form (including granules, powders or suppositories) or in a liquid form (e.g., solutions, suspensions, or emulsions). The compounds can be applied in a variety of solutions. Suitable solutions for use in accordance with the methods are sterile, dissolve sufficient amounts of the peptide, and are not harmful for the proposed application. In this regard, the compounds are very stable but are hydrolyzed by strong acids and bases. The compounds are soluble in organic solvents and in aqueous solutions at pH 5-8.

The active compounds can be subjected to conventional pharmaceutical operations such as sterilization and/or can contain conventional adjuvants, such as preservatives, stabilizers, wetting agents, emulsifiers, buffers, etc.

For administration, the active compounds are ordinarily combined with one or more adjuvants appropriate for the indicated route of administration. The compounds can be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds can be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent can include time delay material, such as glyceryl monostearate or glyceryl distearate alone or with a wax, or other materials well known in the art.

In a further aspect, kits are provided for increasing engraftment of hematopoietic stem and progenitor cells in a subject, wherein the kits comprise an effective amount of the active compounds for increasing engraftment of hematopoietic progenitor cells in a subject, and instructions for using the amount effective of active compound as a therapeutic. In a preferred embodiment, the kit further comprises a pharmaceutically acceptable carrier, such as those adjuvants described above. In another preferred embodiment, the kit further comprises a means for delivery of the active compound to a patient. Such devices include, but are not limited to syringes, matrical or micellar solutions, bandages, wound dressings, aerosol sprays, lipid foams, transdermal patches, topical administrative agents, polyethylene glycol polymers, carboxymethyl cellulose preparations, crystalloid preparations (e.g., saline, Ringer's lactate solution, phosphate-buffered saline, etc.), viscoelastics, polyethylene glycols, and polypropylene glycols. The means for delivery can either contain the effective amount of the active compounds, or can be separate from the compounds, that are then applied to the means for delivery at the time of use.

The growth factors used in the methods are polypeptides. If recruitment or engraftment growth factors are employed, normal routes of in vivo polypeptide administration are preferred, including subcutaneous, intravenous (iv), intraperitoneal (ip), intramuscular (im), and intralymphatic (il). Most preferably in vivo administration of a growth factor is subcutaneous.

Ex vivo use of a growth factor is by direct addition to cultures of hematopoietic stem cells from peripheral blood, umbilical cord blood, or bone marrow in physiological buffer or culture medium. Preferred progenitor cell expansion medium is, for example, minimal essential medium supplemented with autologous serum and antibiotics. Progenitor cell expansion media, comprises one or a plurality of ex vivo growth factors in culture medium, such as minimal essential medium supplemented with autologous serum and possibly antibiotics. Other culture media include, for example, Hanks, McCoys, RPMI 1640 minimal essential media (MEM) and others, and include from 1% to 20% autologous serum and possibly antibiotics.

In some embodiments, in vivo dosages of recruitment or engraftment growth factors range from about 10 μg/kg/day to about 800 μg/kg/day for SF, including but not limited to, 20, 30, 40, 50, 75, 90, 100, 110, 120, 130, 140, 150, 160, 175, 190, 200, 250, 275, 300, 325, 350, 375, 400, 425, 450, 475, 500, 525, 550, 575, 600, 625, 650, 675, 700, 725, 750, 775, and 790; from about 1 μg/kg/day to about 100 μg/kg/day for GM-CSF and IL-3, including but not limited to, 2, 3, 4, 5, 7, 9, 10, 15, 20, 25, 30, 35, 40, 45, 50, 55, 60, 65, 70, 75, 80, 85, 90, 95, 97.5 and 99; and from about 1 μg/kg/day to about 100 μg/kg/day for GM-CSF/IL-3 fusion proteins, including but not limited to, 2, 3, 4, 5, 7, 9, 10, 15, 20, 25, 30, 35, 40, 45, 50, 55, 60, 65, 70, 75, 80, 85, 90, 95, 97.5 and 99. Preferred ex vivo growth factor concentrations in progenitor cell expansion media are from about 1 ng/ml to about 10 μg/ml for SF, including but not limited to, 1.5, 2, 2.5, 3, 3.5, 4, 4.5, 5, 5.5, 6, 6.5, 7, 7.5, 8, 8.5, 9, 9.5, and 9.75, and from about 10 ng/ml to about 200 μg/ml for GM-CSF, IL-3, IL-1 and GM-CSF/IL-3 fusion proteins, including but not limited to, 15, 20, 25, 30, 35, 40, 45, 50, 55, 60, 65, 70, 75, 80, 85, 90, 95, 97.5, 99, 100, 110, 120, 125, 130, 135, 140, 145, 150, 155, 160, 165, 170, 175, 180, 185, 190, and 195.

Progenitor cells can be obtained from human mononuclear cells obtained from bone marrow and peripheral blood. Progenitor cells can be separated from peripheral blood or umbilical cord blood, for example, by density gradient centrifugation such as a Ficoll Hypaque. system. Another means for separating hematopoietic progenitor cells obtained from bone marrow or peripheral blood involves separating with antibodies that recognize a stage-specific antigen on immature human hematopoietic progenitor cells. One example of an antibody recognition method for separating human hematopoietic progenitor cells is described in Civin, U.S. Pat. No. 5,035,994 the disclosure of which is incorporated by reference herein.

Hematopoietic progenitor cells treated ex vivo with growth factors are re-administered to patients by autologous or allogeneic transplantation. Cells are cultured ex vivo in the presence of a growth factor for at least one day and no more than two weeks. Cells can be stored and retain viability either prior to expansion with growth factor or after expansion with growth factor. Cell storage was preferably under cryogenic conditions such as liquid nitrogen. Cultured cells are washed before being administered to the patient. Expanded cells are administered following completion of cytoreductive therapy or up to 72 hours after completion of cytoreductive therapy. Cell administration usually is by infusion over 2 to 5 days. Preferably, from about 10⁷ to about 10⁹ expanded mononuclear cells/kg (approximately 10⁵ expanded progenitor cells/kg) are administered to the patient for an autologous transplantation in conjunction with a Rho modulator.

The compositions and preparations described preferably contain at least 0.1% of active compound. The percentage of the compositions and preparations can, of course, be varied, and can contain between about 2% and 60% of the weight of the amount administered. The amount of active compounds in such pharmaceutically useful compositions and preparations is such that a suitable dosage will be obtained.

### Kits

Kits are also disclosed that are useful in the methods. Such a kit contains an appropriate quantity of active compound, and other components useful for the methods. For example, a kit used to facilitate engraftment of hematopoietic stem cells contains an appropriate amount of the active compound to facilitate engraftment, as well as an amount of the active compound to enhance the engraftment of the stem cells by growth factors. Such a kit can also contain an appropriate amount of a growth factor.

However, it is understood that the present invention is not limited by any particular theory or proposed mechanism to explain its effectiveness in an end-use application.

In effecting treatment of a patient for engraftment of hematopoietic cells, an active agent can be administered in any form or mode, that makes the compound bioavailable in effective amounts, including oral and parenteral routes. For example, the compound can be administered orally, subcutaneously, intramuscularly, intravenously, transdermally, intranasally, rectally, and the like. Oral administration is generally preferred. One skilled in the art of preparing formulations can readily select the proper form and mode of administration depending upon the relevant circumstances.

The pharmaceutical compositions can be formulated for the oral, sublingual, subcutaneous, intravenous, transdermic or rectal administrations in dosage units and in admixture with pharmaceutical excipients or vehicles. Convenient dosage forms include, among those for oral administration, tablets, powders, granulates, and, among those for parenteral administration, solutions especially for transdermal administration, subcutaneous injection, intravenous injection, intraperitoneal injection, intramuscular injection, intrastemal injection, intrathecal injection and infusion techniques.

The dosage can vary widely as a function of the age, weight and state of health of the patient, the nature and the severity of the ailment, as well as of the administration route. These doses can naturally be adjusted for each patient according to the results observed and the blood analyses previously carried out.

In addition, information regarding procedural or other details supplementary to those set forth herein, are described in cited references specifically incorporated herein by reference.

### EXAMPLES

The dominant negative mutant of Rac (Rac2D57N) is associated with a human phagocyte immunodeficiency. Hematopoietic stem cells (HSC) expressing the dominant negative Rac are shown herein to have decreased stem cell engraftment. Likewise, hematopoietic stem cells genetically engineered to be deficient in Rac decrease engraftment. The following examples provide data and protocols for an embodiment of the method of engraftment and dominant negative Rac cells were used for proof of principle. Example 1 identifies the method of producing dominant negative Rac cells.

### Example 1

### Production of Dominant Negative Rac Cells

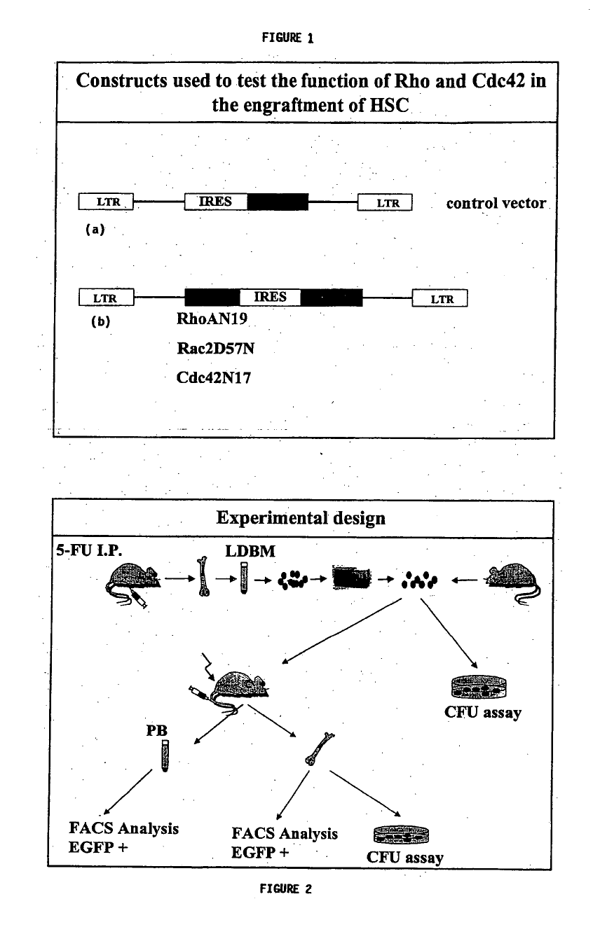

Retrovirus constructs: As shown in FIG. 1, Rac2D57N, RhoAN19 and Cdc42N17 cDNAs were cloned into a modified murine stem cell virus-based bicistronic vector (Williams D A et al. Blood 2000, incorporated by reference in its entirety) at unique EcoRI and XhoI sites. Cells expressing the vectors could be identified because they also expressed enhanced green fluorescence protein (EGFP) and fluoresced green. The viruses were grown and viral supernatant isolated as follows:

Viral supernatant: High titer ecotropic viral supernatant was produced by triple transfection of Phoenix-GP cells with plasmids that express gag (10 μg), ecotropic envelope (3 μg) and the viral construct (8 μg), using Ca²⁺ transfection protocol (Invitrogen, Saint Louis, Mo.). The supernatant was collected in Dulbecco's Modification of Eagle's Medium (DMEM), 10% Fetal Calf Serum (Hyclone Laboratories, Logan, Utah), 2% penicillin/streptomycin at 36 h, 48 h, 60 h and 72 h post transfection and stored at −80 C. Bone marrow cells were isolated an transduced with the virus as follows:

Mouse Low Density Bone Marrow (LDBM) Isolation and Viral Transduction: Whole bone marrow was collected from C57B1/6 animals (Jackson Laboratory, Bar Harbor, Me.), 48 h after treatment with 5-fluorouracil (150 mg/kg of body weight, IP; American Pharmaceutical Partners, Los Angeles, Calif.) and was re-suspended in RPMI medium (Life Technologies, Inc.). Bone marrow cells were obtained from the animals, and low-density, mononuclear bone marrow (LDBM) cells are isolated from fresh bone marrow (BM) by density centrifugation on Histopaque-1083 (Sigma, Carlsbad, Calif.) gradient for 20 min at 1,700 rpm at room temperature. Cells at the interface were collected and washed twice with Roswell Park Memorial Institute (RPMI) Media and then counted on a hemocytometer. LDBM cells (5 million/well) were plated on a six-well non-tissue culture treated plate (Becton Dickinson, N.J., USA) and prestimulated in Iscove's modified Dulbecco's medium (IMDM) (Life Technologies, Inc.) supplemented with 10% fetal calf serum (FCS), 2% penicillin and streptomycin, cytokines (100 ng/ml hG-CSF, 100 ng/ml MGDF, 100 ng/ml SCF (all from Amgen, Thousand Oaks, Calif.) for 48 h at 37° C. in 5% CO₂. Virus infection was performed as previously described (Hanenberg H. et al. Human Gene Therapy, 1997, incorporated by reference in its entirety). Briefly, 2×10⁶ cells were incubated with viral supernatant for 6 h on fibronectin (CH296) (Takara Bio Inc., Otsu, Japan)-coated six-well plates. The infected cells were kept in culture with IMDM, 10% FCS for 48 h. Cells were subsequently analyzed and sorted for green fluorescence protein (GFP) using a Fluorescence Activated Cell Sorting (FACS) (Becton Dickinson, San Jose, Calif.). The bone marrow cells were tested for engraftment as follows:

The transduction efficiency of the cells was tested using the following assay: Colony assay: 2×10⁴ cells re-suspended in 4 mL complete methylcellulose (Metho Cult™ GF M3434, Stem Cell Technologies) were plated in 35 mm grided non-tissue culture dishes (Nalge Nunc Int.) (1 mL/dish). The colonies were scored after 10 days incubation in 5% CO₂ at 37° C. Transduction efficiency was similar among groups with about 40%-50% EGFP positive cells, by FACS-36%-40% EGFP positive CFU. Expression of the transgene in the progenitor cells had no effect on the ability of these cells to form colonies in methylcellulose.

### Example 2

### Analysis of Transgene Expression

Following the production of the dominant negative Rac cells, the transgene expression was tested as follows: Transgene expression: 1×10⁵ transduced and GFP positive BM cells were lysed for 30 min. on ice using 15 μL of 2× lysis buffer (50 mM Tris-HCl, pH 7.5, 10 mM MgCl₂, 200 mM NaCl, 2% Nonidet P-40, 10% glycerol, 2 mM phenylmethylsulfonyl fluoride, 2 μg/ml leupeptin, and 2 μg/ml aprotinin) (all from Roche). The lysate was clarified by centrifugation for 30 min. at 12000 rpm. The supernatant containing total proteins was incubated with 15 μL of Laemmli sample buffer. The proteins were revolved by Sodium Dodecyl Sulphate—Polyacrilamide gel electrophoresis (SDS-PAGE) and transferred to a nitrocellulose membrane (Millipore, Bedford, Mass.). The membrane was blotted using mouse antibodies for total Rac (23A8, 1:2,000, Upstate Biotechnology, Lake Olacid, N.Y.), Cdc42 (1:1,000, BD Transduction Laboratories), and RhoA (1:500, Santa Cruz Biotechnology, Santa Cruz, Calif.). Next the functional activity of the Dominant Negative Rac cells was tested in Example 3 using a standard GTPase assay.

### Example 3

### In Vivo Transplantation and Engraftment Studies

FIG. 2 is a graphical representation of the experimental design showing the design for injecting mice with bone marrow cells. The peripheral blood (PB) cells were tested for EGFP+ cells by FACS analysis and the bone marrow (BM) cells were tested for EGFP+ cells by FACS analysis and by CFU assay.

As shown in FIG. 2, transfected LDBM cells, sorted for GFP expression, were mixed in with fresh isolated bone marrow cells (ratio 7:3) and re-suspended in (Phosphate Buffered Saline) PBS at a concentration of 2×10⁶ cells/mL. 4×10⁵ cells were injected into C57BL/6 mice (8-10 weeks old). Prior to the transplant, the mice were irradiated with 11.75 Gy in split dose (7 Gy and 4.75 Gy) using a Cs¹³⁷ source with a minimum of 3 h between doses. For engraftment studies, 100 μL tail vein blood samples were withdrawn from each transplanted mouse every 4 weeks. Peripheral blood cells were incubated in red blood cell lysis buffer (Pharmingen, San Diego, Calif.) for O min at room temperature. Cells were then washed twice and re-suspended in 1 ml of PBS and 0.2% bovine serum albumin (BSA) (Roche Applied Sciences, Indianapolis, Ind.). The percentages of GFP⁺ cells of each sample were analyzed by flow cytometry. Sixteen weeks post transplant bone marrow and spleen were isolated from the experimental animals. Cells isolated from these organs were incubated in red blood cells lysis buffer, re-suspended in PBS, 0.2% BSA and analyzed by flow cytometry, as described above.

### Example 4

### GTPase activity of the Dominant Negative Rac cells with and without treatment with an agonist

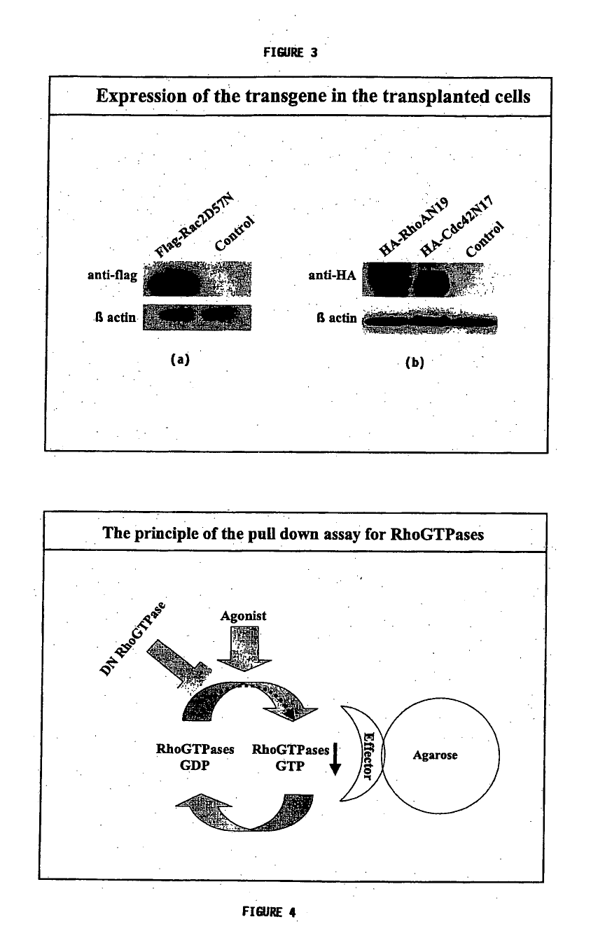

GTPase activity: To determine the level of activated GTPase, GST-effector pull-down assays were performed. NIH 3T3 cell were transduced with retroviral supernatant in the presence of 8 μg/mL polybrene (Sigma, Saint Louis, Mo.). Forty-eight hours after transduction GFP positive cells were sorted and expanded. The cells were subsequently plated at 5×10⁵ cells/10 cm tissue culture dish (Corning) and starved for 16 h in IMDM, 2% penicillin/streptomycin. When treated with the agonist, the cells were then stimulated as follows: for Rac activity Platelet Derived Growth Factor (PDGF) BB (10 ng/mL, PeproTech, Rocky Hill, N.J.) for 1 min., for Rho activity Lysophosphatidic acid (LPA) (5 μg/mL, Sigma) for 5 min., for Cdc42 activity bradikinin (20 μg/mL) for 5 min. After stimulation, the cells were washed with ice-cold PBS and detached from the plate with a disposable cell scraper (Fisher Scientific). The cells were incubated with 2× lysis buffer (50 mM Tris-HCl, pH 7.5, 10 mM MgCl₂, 200 mM NaCl, 2% Nonidet P-40, 10% glycerol, 2 mM phenylmethylsulfonyl fluoride, 2 μg/ml leupeptin, and 2 μg/ml aprotinin), (all from Roche) on ice for 30 min. The lysates were clarified by high speed centrifugation (12000 rpm) for 30 min at 4° C. The total cell extract was incubated with 20 μL of a 50% slurry of agarose beads coated with Rac binding domain (RBD) of p21 activated kinase 1 (Pak1) (for Cdc42 and Rac) and Rhothekin (for Rho) (both from Upstate, Charlottesville, Va.) on a rocker for one hour at 4° C. and then washed three times with washing buffer (25 mM Tris-HCl, pH 7.5, 1 mM DTT, 30 mM MgCl₂, 40 mM NaCl, and 0.5% Nonidet P-40). The beads were centrifuged and the pellets are finally re-suspended in 15 μl of Laemmli sample buffer. Each sample was separated on 12% SDS-polyacrylamide gel electrophoresis and blotted by specific antibodies for total Rac, Cdc42 and RhoA, B, C as above. The secondary antibodies used were horseradish peroxidase-conjugated (1:2,500, New England Biolabs). The immunoblots were detected by New England Biolabs Luminol kit and Kodak Biomax film (FIGS. 3, 5 and 7).

FIG. 3 is an immunoblot showing expression of the transgene in the transplanted cells where (a) shows the Flag-Rac2D57N and control vectors and (b) shows the HA-RhoAN19, HA-Cdc42N17 and control vectors.

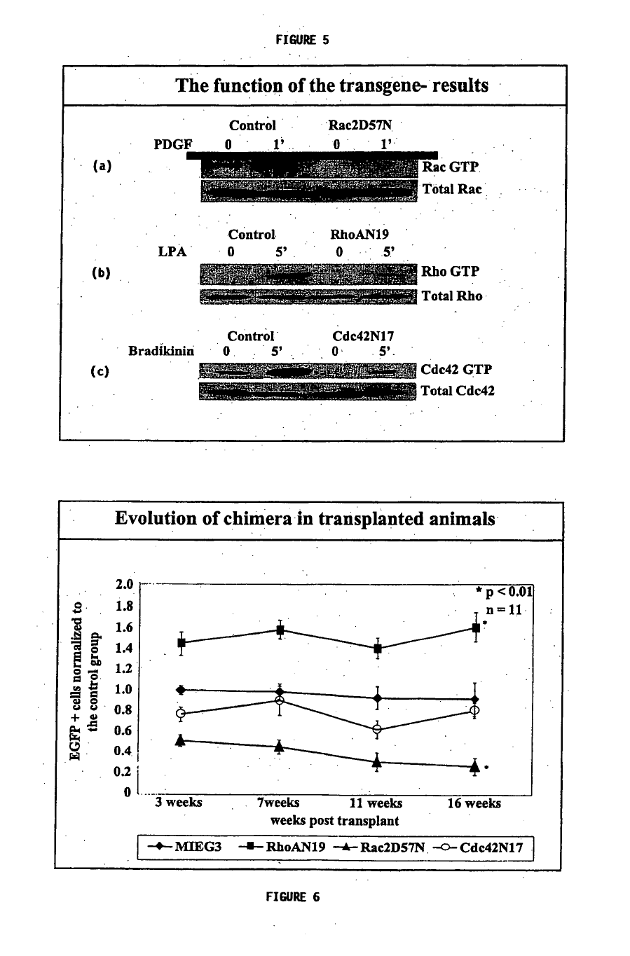

FIG. 5 shows Immunoblot results of the function of the transgenes in peripheral blood cells stimulated with agonists where (a) shows the effects of PDGF stimulation on RacGTPase for control and Rac2D57N cells; (b) shows the effects of LPA stimulation on RhoGTPase for control and RhoAN19 cells; and (c) shows the effects of bradikinin stimulation on Cdc42GTP for control and Cdc42N17 cells.

### Example 5

### Expression of the Trangene in the Bone Marrow of Mice

FIG. 7 is an immunoblot showing the expression of the transgene in the bone marrow of the mice at 16 weeks post-transplantation where (a) shows the Flag-Rac2D51N and control vectors and (b) shows the HA-RhoAN19, HA-Cdc42N17 and control vectors. The immunoblot was prepared as in Example 4.

FIG. 4 is a graphical representation of the pull-down assay for RhoGTPases as described herein where a dominant negative (DN) form of RhoGTPase was used to inhibit the activation of the RhoGTPase from the GDP form to the GTP form. Agarose beads coated with an effector were used to concentrate the active form of the GTPase.

### Example 6

### Effect of Rho and Rac on Stem Cell Engraftment

The ability of hematopoietic progenitor cells to form colonies was analyzed in the presence of various transgenes expressing normal and mutated forms of GTPases. The transgenes were expressed in the cells and the cells engrafted as in Examples 1-5.

FIG. 6 shows that the evolution of the chimera in transplanted animals. The EGFP+ peripheral blood cells were analyzed at 3, 7, 11, and 16 weeks for MIEG3, RhoAN19, Rac2D57N, and Cdc42N17 transplanted animals and normalized to the control cells at 3 weeks and where p<0.01 and n=11. This clearly shows that the expression of dominant negative Rac was detrimental for engraftment. While expression of dominant negative Rho was advantageous for engraftment.

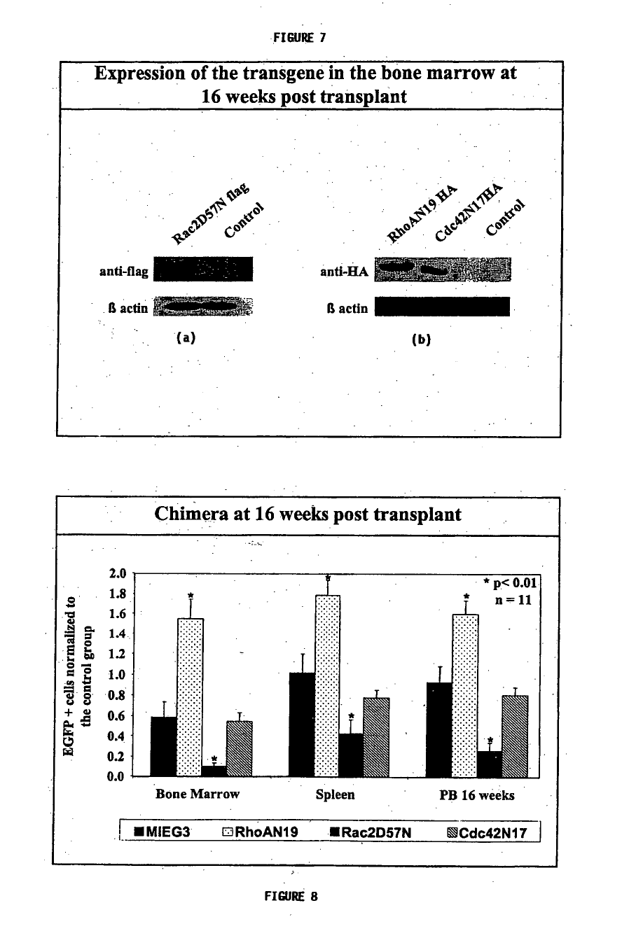

FIG. 8 is a graphical representation of the same experiment but using EGFP+ cells from bone marrow, spleen and peripheral blood at 16 weeks. Cells expressing MIEG3, RhoAN19, Rac2D57N, and Cdc42N17 and control vectors were normalized to the control cells at 3 weeks where p<0.01 and n=11.

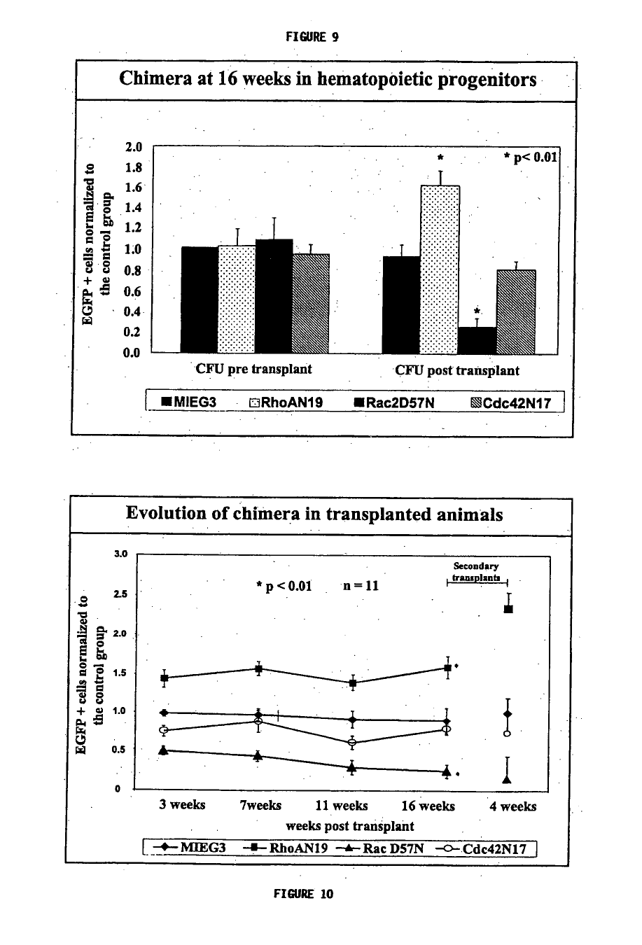

The same analysis was performed on progenitor cells and peripheral blood cells from transplanted animals in FIGS. 9 and 10 as follows:

FIG. 9 shows EGFP+ progenitor cells (CFU's) pre-transplant and at 16 weeks expressing MIEG3, RhoAN19, Rac2D57N, and Cdc42N17 as compared to control vectors normalized to the control cells at 3 weeks where p<0.01.

FIG. 10 shows EGFP+ peripheral blood cells at 3, 7, 11, and 16 weeks for MIEG3, RhoAN19, Rac2D57N, and Cdc42N17 transplanted animals normalized to the control cells at 3 weeks and where p<0.01 and n=11. In addition, the graph shows the results of a secondary transplant wherein bone marrow cells harvested at 16 weeks post-transplantation were used in a secondary transplantation and measured for EGFP activity after 4 weeks.

The above experiments show that the ability of hematopoietic progenitor cells to form colonies was not affected by expression of Rac2D57N, RhoAN19 or Cdc42N17 in the cells (FIG. 9). However, the expression of dominant negative Rac was detrimental for the stem cell engraftment (FIGS. 6, 8, 9, and 10). In contrast, expression of dominant negative Rho proved to be advantageous for HSC engraftment (FIGS. 6, 8, 9, and 10).

In this experimental setting Cdc42 did not seem to play a major role in HSC engraftment while Rho GTPases played a critical role in the engraftment of hematopoietic stem cells. While stem cells were mobilized from the bone marrow by transiently decreasing the activity of Rac, transient down regulation of the activity of Rho was beneficial for the engraftment of HSC. Further experiments, methods, and treatments showing the effect on mobilization of the hematopoietic cells can be found in U.S. patent application Ser. No. ______, filed Aug. 13, 2004, entitled “Mobilization of Hematopoietic Cells” (attorney docket number CHMC20.001A), based on U.S. Provisional application 60/494,718, filed Aug. 13, 2003 entitled “Mobilization of Hematopoietic Cells”, herein incorporated by reference in its entirety.

The various methods and techniques described above provide a number of ways to carry out the methods. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as can be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

1. A method for enhancing bone marrow engraftment in an subject in need thereof comprising administering to the subject (i) a bone marrow graft and (ii) a Rho modulator, wherein the Rho modulator is administered in an amount effective to promote engraftment of the bone marrow in the subject.

2. The method of claim 1, wherein the Rho modulator comprises a modulator of at least one RhoGTPase selected from the group consisting of RhoA, RhoB, and RhoC.

3. The method of claim 1, wherein the Rho modulator comprises a modulator of RhoA.

4. The method of claim 1, wherein the Rho modulator comprises a HMG CoA reductase inhibitor.

5. The method of claim 4, wherein the HMG CoA reductase inhibitor comprises a statin.

6. The method of claim 5, wherein the statin comprises at least one compound selected from the group consisting of lovastatin, simvastatin, pravastatin, fluvastatin, atorvastatin, and cerivastatin.

7. The method of claim 5, wherein the statin comprises atorvastatin.

8. The method of claim 4, wherein said HMG-CoA reductase inhibitor comprises an oxysterol.

9. The method of claim 8, wherein said oxysterol comprises 25-hydroxycholesterol.

10. The method of claim 1, wherein the Rho modulator comprises a combination of an HMG CoA reductase inhibitor with an inhibitor of geranylgeranyl-protein transferase or farnesyl transferase.

11. The method of claim 1, wherein the Rho modulator comprises one or more agents selected from the group consisting of: a farnesyl protein transferase (FPTase) inhibitor, a prenyl-protein transferase inhibitor, and a geranylgeranyl-protein transferase inhibitor.

12. The method of claim 1, wherein the Rho modulator comprises a toxin selected from the group consisting of: toxin A, toxin B, C. sordellii lethal toxin (LT), botulinum toxin, and Staphylococcal toxin EDIN.

13. The method of claim 1, wherein the Rho modulator comprises a geranylgeranyl transferase inhibitor.

14. The method of claim 1, wherein the Rho modulator comprises a Rho activated kinase.

15. The method of claim 1, wherein the Rho modulator comprises an antisense compound targeted to a nucleic acid molecule encoding a member of the human Rho family of small GTP binding proteins, wherein the antisense compound inhibits the expression of the member of the human Rho family.

16. The method of claim 15, wherein the antisense compound comprises an antisense oligonucleotide.

17. The method of claim 16, wherein the antisense compound is from 8 to 30 nucleobases in length.

18. The method of claim 15, wherein the member of the human Rho family of small GTP binding proteins is selected from the group consisting of: RhoA, RhoB, RhoC, and RhoG.

19. The method of claim 15, wherein the member of the human Rho family of small GTP binding proteins is RhoA.

20. The method of claim 1, wherein the Rho modulator comprises a Rho family antagonist.

21. The method of claim 20, wherein the Rho family antagonist inhibits a Rho regulatory pathway via interaction with a GTP/GDP cycle.

22. The method of claim 21, wherein the interaction with the GTP/GDP cycle involves a GTP/GDP exchange protein (GEP); a GDP dissociation inhibitor (GDI); or a GTPase activating protein (GAP) to regulate Rho activity.

23. The method of claim 1, wherein the Rho modulator promotes stem cell engraftment by inhibiting one or more Rho family members in a stem cell or a progenitor cell.

24. The method of claim 23, wherein the Rho modulator is selected from the group consisting of ADP-ribosyl transferase C3, toxin A and toxin B.

25. The method of claim 23, wherein the Rho modulator comprises one or more biologically active fragments selected from the group consisting of: ADP-ribosyl transferase C3, analogs of ADP-ribosyl transferase C3 and derivatives of ADP-ribosyl transferase C3.

26. The method of claim 1, wherein the Rho modulator comprises a genetically mutated form of Rho that promotes stem cell engraftment by inhibiting one or more Rho family members in stem cells.

27. The method of claim 26, wherein the genetically mutated form comprises a mutation in the effector domain and prevents or interferes with GTP exchange.

28. The method of claim 1, wherein the Rho modulator comprises a dominant negative Rho protein.

29. The method of claim 1, wherein the Rho modulator comprises a nucleic acid expressing a dominant negative Rho protein.

30. The method of claim 29, wherein the nucleic acid comprises an expression construct comprising a viral vector.

31. The method of claim 30, wherein the viral vector comprises selected from the group consisting of: a retroviral vector, an adenoviral vector and an adeno-associated viral vector.

32. The method of claim 31, wherein the adenoviral vector comprises a replication-deficient adenoviral vector.

33. The method of claim 1, wherein the Rho modulator comprises a vector comprising a polynucleotide encoding a dominant negative Rho protein operably linked to an organ-specific promoter.

34. The method of claim 1, wherein the Rho modulator comprises an antisense compound targeted to a nucleic acid molecule encoding a member of the human Rho family of small GTP binding proteins, and wherein the antisense compound inhibits the expression of the member of the human Rho family.

35. The method of claim 34, wherein the antisense compound comprises an antisense oligonucleotide.

36. The method of claim 35, wherein the antisense compound is from 8 to 30 nucleobases in length.

37. The method of claim 34, wherein the member of the human Rho family of small GTP binding proteins is selected from the group consisting of: RhoA, RhoB, RhoC, and RhoG.

38. The method of claim 37, wherein the member of the human Rho family of small GTP binding proteins is RhoA.

39. The method of claim 1 wherein the bone marrow graft comprises hematopoietic stem or progenitor cells.

40. The method of claim 1 wherein the Rho modulator is administered by intravenous injection or by injection directly to the site of intended activity.

41. The method of claim 1 wherein at least one of (i) the Rho modulator and (ii) the bone marrow graft is administered intravenously.

42. The method of claim 1 wherein at least one of (i) the Rho modulator and (ii) the bone marrow graft is administered by injection directly to the site of intended activity.

43. The method of claim 1 wherein the bone marrow graft is autologous.

44. The method of claim 1 wherein the Rho modulator is administered concurrently with the bone marrow graft.

45. The method of claim 1 wherein the Rho modulator is administered to the subject prior to administration of the bone marrow graft.

46. The method of claim 1 wherein the Rho modulator inhibits the activity of a RhoGTPase by at least 50%.

47. The method of claim 46 wherein the activity of the RhoGTPase is reduced by at least 75%.

48. The method of claim 47 wherein the activity of the RhoGTPase is reduced by at least 90%.

49. A method for autologous hematopoietic cell transplantation in a subject receiving cytoreductive therapy, comprising: (a) removing hematopoietic stem or progenitor cells from the subject prior to cytoreductive therapy; (b) expanding the hematopoietic stem or progenitor cells ex vivo with a cell expanding amount of a Rho modulator to provide a cellular preparation comprising an expanded population of hematopoietic stem or progenitor cells; (c) administering the cellular preparation to the subject following cytoreductive therapy; and (d) administering a Rho modulator, wherein the Rho modulator is administered in an amount and dosage regime effective to promote engraftment of the cells in the subject.

50. The method of claim 49 further comprising the step of subsequently treating the patient in vivo with an engraftment growth factor for 3 to 21 days following administrating the cellular preparation to the patient, wherein the engraftment growth factor is selected from the group consisting of GM-CSF, IL-3, SF, GM-CSF/IL-3 fusion proteins and combinations thereof.

51. The method of claim 49 wherein the hematopoietic stem or progenitor cell are obtained from peripheral blood.

52. The method of claim 49 wherein the hematopoietic stem or progenitor cells are obtained from bone marrow.

53. The method of claim 49, wherein the hematopoietic stem cells are derived from human umbilical cord blood or human bone marrow or are obtained by purifying whole blood.

54. The method of claim 49, wherein the hematopoietic stem cells are grown ex vivo under conditions that increase the total number CD34+/CD38− cells.

55. The method of claim 49, further comprising administering a hematopoietic stem or progenitor cell-stimulating amount of at least one growth factor prior to, subsequent to, or concurrently with, administration of the Rho modulator.

56. The method of claim 55, wherein the growth factor is selected from the group consisting of CSF-1, GM-CSF, SF, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF, FGF, TPO and combinations thereof.

57. The method of claim 56, wherein the growth factor comprises G-CSF.

58. The method of claim 57, wherein the effective amount of the Rho modulator is administered in a series of doses.

59. A method of testing for an agent that modulates the binding between a guanine nucleotide exchange factor and a RhoGTPase comprising: contacting a polypeptide comprising a GTPase, or a biologically-active fragment thereof, with an agent suspected of modulating the activity of the RhoGTPase; and detecting the activity of the GTPase.

60. The method of claim 59, wherein the RhoGTPase is RhoA.
